(19) United States
(12) Patent Application Publication
(10) Pub. No.: US 2006/0182736 A1
(43) Pub. Date: Aug. 17, 2006

(54) Transducible dna-binding proteins

(76) Inventors: Jin-Soo Kim, Daejeon (KR); Hyun-Chul Shin, Daejeon (KR); Heung-Sun Kwon, Daejeon (KR)

Correspondence Address: SUGHRUE MION, PLLC, 2100 PENNSYLVANIA AVENUE, N.W., SUITE 800, WASHINGTON, DC, 20037, US

(21) Appl. No.: 10/559,806

(22) PCT Filed: Jun. 10, 2004
(86) PCT No.: PCT/KR04/01385

§ 371 Date: Dec. 8, 2005

(30) Foreign Application Priority Data
Jun. 10, 2003 (US) 60477459

(51) Int. Cl. A61K 38/46; C07H 21/04; C12P 21/06; C12N 9/64; A61K 33/32; C12N 5/08

(52) U.S. Cl. 424/94.65; 435/69.1; 435/226; 435/366; 435/320.1; 536/23.2; 424/641

(57) ABSTRACT

Disclosed is a chimeric protein comprising: a) a plurality of zinc finger domains; and b) a heterologous protein transduction domain, effective for translocating the protein across a cellular membrane. The chimeric protein can be efficiently transduced into cultured eukaryotic cells in which they regulate specific target genes. Thus, artificial transcription factors that include a PTD can be used to produce protein drugs that regulate endogenous genes and protein drugs that alter cell behavior in vitro and in vivo.

### CROSS-REFERENCE TO RELATED APPLICATIONS

This application claims priority to U.S. Ser. No. 60/477,459, filed Jun. 10, 2003, the contents of which are hereby incorporated by reference in its entirety.

### BACKGROUND

This invention relates to DNA binding proteins. Many transcription factors that bind DNA have a modular structure that includes a DNA-binding domain and an effector domain. There are many types of DNA-binding domains. Zinc finger domains are one of the most abundant types of DNA-binding domains among eukaryotic transcription factors. Because zinc finger domains are modular, these domains are ideal for generating artificial DNA-binding proteins with useful properties.

### SUMMARY

This disclosure includes evidence that exogenous chimeric zinc finger proteins that include a protein transduction domain (PTD) can be efficiently transduced into cultured mammalian cells in which they regulate specific target genes. Thus, artificial transcription factors that include a PTD can be used to produce protein drugs that regulate endogenous genes and protein drugs that alter cell behavior in vitro and in vivo. One advantage of protein drugs is that they have a finite produrance. Their concentration and consequently their effect can be carefully controlled and limited.

Because zinc finger domains chelate a zinc ion, it was unclear prior to the present disclosure whether zinc finger domains could translocate across biological membranes and retain their functionality. Unfolding of the domain during translocation may cause the zinc ion to be released and lost. Conversely, the folded state may prevent translocation. Further, exposure of zinc finger domains to the oxidizing conditions in the extracellular environment may cause detrimental disulfide bond formation between cysteine residues, thereby disabling DNA binding activity. We have discovered that zinc finger domain can, in fact, be translocated across biological membranes. Moreover, we have found that, after translocation, these domains are functional and can regulate endogenous genes in a cell.

Accordingly, in one aspect, the invention features a protein (e.g., a chimeric and/or isolated) that includes: a) a zinc finger domain; and b) a heterologous protein transduction domain. The protein can include a plurality of zinc finger domains, e.g., two, three, four, five, or six zinc finger domains, or at least two, three, four, five, or six zinc finger domains. In one embodiment, the protein includes an array of zinc finger domains.

The protein can also include one or more of the following features: a nuclear localization signal, a dimerization domain, a cell targeting domain, cell surface protein binding domain, a purification handle and an effector domain. The protein can also include one or more non-peptide backbone bonds or an artificial amino acid. The cell targeting domain can include an immunoglobulin variable domain, a growth factor, a cell binding domain of a viral protein, or a cell binding domain of an extracellular protein. The purification handle can include an amino acid sequence that is free of cysteines and can chelate metal, e.g., penta- or hexa-histidine.

In one embodiment, the zinc finger domain is naturally occurring, e.g., human. In another embodiment, it is not naturally occurring. For example, it is possible to humanize zinc finger domains, e.g., by altering many non-DNA contacting residues to be identical to corresponding residues in a human zinc finger domain. In one embodiment, the protein transduction domain and the zinc finger domain are components of the same polypeptide chain. The protein transduction domain and the zinc finger domain can be separated by at least 10, 20, or 50 amino acids. For example, they can be separated by one or more of: a flexible linker, a protease cleavage site for a site specific protease (e.g., a site specific intracellular protease), or a functional domain.

In another embodiment, they are components of separate polypeptide chains. For example, the chains can be attached by a reducible bond (e.g., a disulfide bond) or another bond which is cleaved upon cell entry.

In one embodiment, the protein transduction domain includes a viral sequence, e.g., from a virus that naturally infects humans, e.g., an HIV virus. An exemplary protein transduction domain is the HIV TAT protein transduction domain, e.g., the amino acid sequence: YGRKKRRQRRR (SEQ ID NO:1). The viral sequence can be between 5-50, or 8-20 amino acids in length. In another embodiment, the protein transduction domain includes a mammalian sequence, e.g., a sequence from a human protein. In another embodiment, the protein transduction domain includes an artificial sequence, e.g., a sequence identified as a transduction domain from a display library. In an embodiment, the protein transduction domain is a cell-specific transduction domain. The term “heterologous” indicates that the protein transduction domain and the zinc finger domain are not derived from the same protein. For example, the zinc finger domain can be artificial, whereas the protein transduction domain can be derived from, e.g., a viral protein.

In one embodiment, the protein can regulate transcription of at least one endogenous gene in a cell after the protein is contacted with the exterior of the cell. The protein may be internalized by crossing a plasma membrane of the cell, e.g., at the cell surface or after vesicle formation. For example, the protein can regulate transcription of at least one endogenous gene in a cell, but fewer than 1%, 0.01% or 0.001% of the genes in the cell after the protein is contacted with the exterior of the cell. The number of genes regulated by the protein can be determined, e.g., using a nucleic acid microarray. In many particular cases, the protein regulates the same genes as a protein that lacks the protein transduction domain, but is otherwise identical.

Typically the protein can translocate from the extracellular milieu into a mammalian cell in the absence of another factor, e.g., an artificial factor such as a cell permeabilization reagent (e.g., a detergent).

In one embodiment, the protein includes a conditional domain that regulates the function of another domain of the protein such that the function depends on presence or absence of an exogenous compound. For example, the conditional domain binds to a small molecule, e.g., a steroid, FK506, and so forth. A “small molecule” is a molecule that has a molecular weight of less than 4 kDa. For example, the conditional domain can include an FK506 binding domain.

In one embodiment, the protein can be transduced into at least 50, 75, 80, 90, or 95% of cultured human embryonic kidney (HEK) 293 cells in an assay in which the cells are at 3×10⁵/ml and the protein is present in the extracellular medium at a concentration of 100 μg/ml, or at a concentration of less than 100 μg/ml, 50 μg/ml, 5 μg/ml, or 0.5 μg/ml.

In a related embodiment, the invention features an isolated protein that includes: a) an artificial DNA binding domain that binds to a naturally occurring target site in a cell with an affinity (K_{d}) of less than 75, 50, 25, 20, 10, 5, 2.5, 1, 0.5, or 0.05 nM; b) a heterologous protein transduction domain that can cause the isolated protein to enter the cell across an external membrane of the cell; and c) a nuclear localization signal.

The invention also features pharmaceutical compositions that include a transducible protein described herein and a pharmaceutically acceptable carrier. The composition can further includes an agent which stabilizes the redox potential of the composition, e.g., a reducing agent or reductant in an amount effective to decrease disulfide bond formation between cysteine residues of the zinc finger domain of the protein, e.g., dithiothreitol (DTT) or β-mercaptoethanol. For example, the composition can include glutathione. In one embodiment, the composition further includes zinc, e.g., a zinc salt such as zinc chloride or zinc acetate. Useful concentrations of zinc include 1 μM to 5 mM, 1 μM to 500 μM, 1 μM to 200 μM, 0.05 μM to 50 μM, and 0.5 μM to 30 μM. In another embodiment, the composition is substantially free of zinc, e.g., zinc concentration is less than 0.5 mM.

The transducible protein can be expressed in E. coli or another prokaryotic cell, e.g., as inclusion bodies or as a secreted protein. In another example, the transducible protein is expressed in a eukaryotic cell, e.g., a mammalian cell, plant cell, or yeast cell. The transducible protein can be purified, e.g., using at least one or two purification steps, e.g., at least two chromatography steps, e.g., an affinity chromatography step and an ion exchange chromatography step. The protein can be sufficiently pure that it is stable in human tissue culture cells (e.g., HEK293) cells for at least 0.5, 1, 2, 3, 6, 12, 24, 48, or 60 hours. The protein may be said to be stable if it is detectable after the requisite time period, e.g., not susceptible to substantial degradation. The protein can be at least sufficiently pure that it is the only detectable band on a Coomassie gel when about 10 μg of protein are loaded in a lane.

The invention also features a method that includes administering the pharmaceutical composition to a subject, e.g., in an amount effective to alter gene expression in a cell of a subject or in an amount effective to cause a phenotypic change in the subject. The method can include administering the composition in a plurality of doses, e.g., at least two, three, six, ten, or twenty doses at separate times or continuously (e.g., using a medical device or intravenous delivery system). In cases where the composition is administered using a plurality of doses, the doses can separated, e.g., by at least 12, 24, 48, 60, 72, or 96 hours, e.g., by between 24-96 hours, or by at least 120 hours. For example, the method can be used to reduce angiogenesis in a subject, e.g., a subject having a neoplastic disorder or suspected of having a neoplastic disorder. The protein can specifically bind to a site in the VEGF-A gene and may further include a repression domain, e.g., that can repress the VEGF-A gene. The method can also be used to treat any disorder described in U.S. Ser. Nos. 10/314,609 and 10/669,861, e.g., using the zinc finger proteins described therein that are physically associated with a protein transduction domain. Such proteins can be administered, e.g., in a plurality of doses, e.g., separated in time as described above.

In another aspect, the invention features a method of regulating an endogenous gene. The method includes contacting a cell with a polypeptide that includes: a) a DNA-binding domain (e.g., one or more zinc finger domains); and b) a heterologous protein transduction domain. The method can further include detecting the polypeptide 12, 24, 48, 60, 72, or 96 hours after the contacting. The cell can be in vitro or in vivo.

The invention also features a nucleic acid that includes a coding sequence that encodes a polypeptide described herein. For example, the coding sequence encodes a polypeptide that includes a) a zinc finger domain; and b) a protein transduction domain, heterologous to the zinc finger domain. The polypeptide can also include a signal sequence, e.g., at the N-terminus to direct secretion of the encoded polypeptide. The signal sequence can include a processing site, e.g., for signal peptidase to remove the signal sequence. In another example, the nucleic acid includes more than one coding sequence, e.g., a first coding sequence encoding a first polypeptide chain that includes a DNA binding array and a second coding sequence encoding a second polypeptide chain that includes a protein transduction domain. The invention also features a host cell that contains a nucleic acid that includes a coding sequence that encodes a polypeptide described herein. For example, the coding sequence encodes a polypeptide that includes a) a zinc finger domain; and b) a protein transduction domain, heterologous to the zinc finger domain. The polypeptide can also include a signal sequence, and the host cell can be capable of secreting the polypeptide, e.g., after processing of the signal peptide, to thereby release a chimeric DNA binding protein. The host cell is useful, e.g., to produce the polypeptide for inclusion in a pharmaceutical composition. The host cell can also itself be used as a therapeutic. For example, the host cell (e.g., a compatible fibroblast or hematopoietic cell) can be introduced into a subject to thereby provide a chimeric DNA binding protein to the subject.

It is also possible to deliver a nucleic acid that encodes a polypeptide that comprises a signal sequence, a plurality of zinc finger domains, and a protein transduction domain to cells, e.g., cells in a subject. The nucleic acid can be operably linked to a tissue specific promoter, e.g., an epithelial cell specific, B- or T-cell specific promoter, and so forth. Method of delivering nucleic acids are known. See, e.g., Hollingsworth (1999) Lancet. 1999 April; 353 Suppl 1: SI19-20; Kremer (1995) British Medical Bulletin 51(1):31-44 and Anderson (1992) Science 256:808-813.

In another aspect, the invention features a method of modulating expression of a gene. The method includes: (1) providing a protein that includes: a) a DNA binding domain that specifically recognizes a target site associated with a gene; and b) a protein transduction domain that is heterologous to the DNA binding domain, and (2) contacting the protein with a cell under conditions that enable the protein to enter the cell and regulate expression of the gene. In one embodiment, the DNA binding domain can include one or a plurality of zinc finger domains. The protein can include an effector domain. The protein can include other features described herein. In one embodiment, the amount of protein used can be less than 100 μg/ml, 50 μg/ml, 0.5 μg/ml, or 5 ng/ml.

In one embodiment, after the contacting, expression of the gene is altered (e.g., increased or decreased) by a factor of at least 0.5, 1, 2, 2.5, 5, 10, 20, or 100. Some proteins may alter the expression of a plurality of genes. In some cases, some or all of these genes are also associated with the target site. The target site can be within 10, 5, 3, or 1 kb or 500, 300, or 200 bp of the start site for transcription of the gene, e.g., upstream or downstream of the start site. In one embodiment, the target site overlaps by at least one base pair with a regulatory site of the gene, e.g., a site bound by an endogenous factor. Target sites can also be present in introns or coding regions. In certain embodiments, it is not necessary to have knowledge of the particular target site bound by the protein. For example, other tests of specificity and functionality can be used to indicate that the protein can regulate expression of a gene.

In one embodiment, the method further includes evaluating the cell. For example, cell can be evaluated for expression of the gene. In another example the cell is evaluated for a phenotype, e.g., a phenotype that depends on expression or repression of the gene.

In one embodiment, the method further includes introducing the cell into a subject. For example, if the protein reduces MHC protein expression, the cell can be used for a temporary transplant into a subject without concern for MHC compatibility.

In another aspect, the invention features a method of modulating a phenotype of a cell. The method includes: (1) providing a protein that includes: a) a DNA binding domain; and b) a protein transduction domain that is heterologous to the DNA binding domain, and (2) contacting the protein with a cell under conditions that enable the protein to enter the cell and regulate the phenotypic state of the cell. In one embodiment, the DNA binding domain can include one or a plurality of zinc finger domains. The protein can include an effector domain. The protein can include other features described herein. In one embodiment, the amount of protein used can be less than 100 μg/ml, 50 μg/ml, 0.5 μg/ml, or 5 ng/ml. In one embodiment, the step of providing includes identifying the protein using a phenotypic screen as a protein that can modulate the phenotype of a cell.

In one embodiment, after the contacting, a quantifiable trait of the cell is altered (e.g., increased or decreased) by a factor of at least 0.5, 1, 2, 2.5, 5, 10, 20, or 100. In one embodiment, the method further includes evaluating the cell. For example, cell can be evaluated for expression of one or more genes or for one or more proteins. In another example the cell is evaluated for a phenotype, e.g., a phenotype that depends on expression or repression of the gene.

In one embodiment, the method further includes introducing the cell into a subject. For example, if the protein reduces MHC protein expression, the cell can be used for a temporary transplant into a subject without concern for MHC compatibility. In another example, if the protein increases insulin expression, the protein can modify cells in a subject to increase insulin levels in the subject or cells modified by the protein can be administered to the subject to increase insulin levels.

In another aspect, the invention features a method of treating a subject having, or at risk for having, a neoplastic disorder. The method includes administering to the subject a composition that includes a transducible protein that can regulate (e.g., inhibit) a cancer promoting gene, e.g., an oncogene or VEGF-A. For example, the transducible protein includes a DNA binding domain such as an array of zinc finger domains, a protein transduction domain, and optionally an effector domain. The protein can be administered in an amount effect to reduce risk of the neoplastic disorder, to reduce growth of a tumor, to reduce angiogenesis, or to ameliorate at least one symptom of the neoplastic disorder. The reduction can be a detectable or statistically significant reduction. For example, the subject is a human subject, e.g., an adult or juvenile. For example, the subject can have a carcinoma or sarcoma.

In another aspect, the invention features a method of altering gene expression in a cell of a subject. The method includes: providing a chimeric DNA binding protein that comprises a DNA binding domain and a heterologous protein transduction domain, the DNA binding protein being able to regulate transcription of an endogenous gene in a cell of the subject; and administering a first dose of the DNA binding protein to a subject. The method can further include administering a second dose of the DNA binding protein to the subject. For example, the first and second dose are separated by at least about 6, 12, 18, 24, 48, 96 or 120 hours. In one embodiment, the first dose is at least 10, 25, 40, 50, or 80% less than the second dose. In another embodiment, the first dose is the same as the second dose. In still another embodiment, the first and the second doses are the same.

In one embodiment, the subject has or is suspected of having a neoplastic disorder, and the DNA binding protein further comprises an effector domain such that the DNA binding protein modulates transcription of a gene that regulates angiogenesis (e.g., VEGF-A) in cells of the subject.

In another aspect, the invention features a method that includes: contacting the cell with a dose of a DNA binding protein that comprises a DNA binding domain and a protein transduction domain. The protein is able to regulate transcription of an endogenous gene in a cell and the dose is effective to regulate transcription of the endogenous gene for at least 6, 12, 18, 24, 48, 96 or 120 hours. The method can further include, at least 6, 12, 18, 24, 48, 96 or 120 hours after the contacting, contacting the cell with a second dose of the DNA binding protein.

In another aspect, the invention features a mammalian cell that contains an exogenous polypeptide, but not a nucleic acid that encodes the exogenous polypeptide, wherein the exogenous polypeptide includes a DNA binding domain and a protein transduction domain that is heterologous to the DNA binding domain. The exogenous polypeptide may be functional to regulate transcription of a selected subset of endogenous genes in the cell for at least 6, 12, 24, 36, 48, or 96 hours (e.g., between 12-96 or 48-96) after introduction of the exogenous polypeptide into the cell. In one embodiment, the DNA binding domain includes a zinc finger domain, e.g., a plurality of zinc finger domains. For example, the zinc finger domain is a naturally occurring zinc finger domain, e.g., a human zinc finger domain. The mammalian cell can be a human cell, e.g., a fibroblast, hematopoietic, neuronal, endothelial, or epidermal cell. The mammalian cell can be characterized by a phenotypic trait which would be absent or altered if the mammalian cell did not include the exogenous polypeptide. For example, the proliferative state of the mammalian cell can depend on the exogenous polypeptide. The mammalian cell can be present in a subject mammal (e.g., as one of the mammal's own cells or as an exogenously introduced cell) or in culture. For example, the mammalian cell can be present in a human. An exemplary mammalian culture cell is HEK293 cell. In another example, the mammalian cell is a primary cell that can be used to treat a subject. In one embodiment, the exogenous polypeptide suppresses expression of an MHC protein.

In another aspect, the invention features a non-human mammal that includes one or more modified cells. The cells are modified by introduction of a polypeptide that includes a DNA binding domain and a protein transduction domain that is heterologous to the DNA binding domain. The modified cells do not contain a nucleic acid encoding the polypeptide. For example, the mammalian may have been treated with a pharmaceutical composition that includes the exogenous polypeptide or with cells that were contacted with the exogenous polypeptide. The exogenous polypeptide can be functional in the cells for at least 6, 8, 12, 20, 24, 36, 48, or 96 hours after being introduced into the cells. The polypeptide can be a polypeptide described herein.

In another aspect, the invention features a method that includes: providing a nucleic acid that includes a first sequence encoding a polypeptide including a DNA binding domain that binds to a specific DNA site with an affinity of less than 75, 50, 25, 20, 10, 5, 2.5, 1, 0.5, or 0.05 nM; and modifying the nucleic acid to include a second sequence that encodes a polypeptide including a protein transduction domain such that the first and second sequences are in frame and encode a fusion polypeptide that includes the DNA binding domain and the protein transduction domain. The method can be used to prepare a transducible transcription factor.

The step of modifying can include one or more of: ligation, recombination in vitro or in vivo, and PCR amplification. For example, the step of modifying includes PCR amplification using an oligonucleotide which anneals to a region of the first sequence or a complement thereof and a region of the second sequence or a complement thereof.

The method can further include introducing the modified nucleic acid into a cell (e.g., a prokaryotic or eukaryotic cell) and maintaining the cell under conditions in which the modified nucleic acid is expressed and the fusion polypeptide is produced.

The method can further include contacting the fusion polypeptide with a cell that does not include the modified nucleic acid. The method can further include purifying the fusion polypeptide away from other contents of the cell, e.g., cell membranes. The method can further include isolating medium surrounding the cell and, optionally, processing the medium in order to obtain the polypeptide in a form more concentrated than in the medium. The method can further include combining the fusion polypeptide with a pharmaceutically acceptable carrier other than water. The composition may further include water.

In another aspect, the invention features a method of preparing a transducible DNA binding polypeptide. The method includes: providing a host cell that contain a nucleic acid including 1) a coding sequence that encodes a polypeptide that includes a) a zinc finger domain; and b) a heterologous protein transduction domain, and 2) a promoter operably linked to the coding sequence; expressing the nucleic acid in the host cell under conditions in which the polypeptide is synthesized; and isolating the polypeptide from the host cell or from medium surrounding the host cell.

In another aspect, the invention features a library of nucleic acids, the library including a plurality of nucleic acids wherein each nucleic acid of the plurality encodes a polypeptide including a zinc finger domain and a protein transduction domain. The nucleic acids of the plurality vary at positions such that the respective nucleic acids encode polypeptides that have different zinc finger domains relative to one another. For example, the plurality includes at least 10², 10⁴, 10⁶, or 10⁸ different members. In one embodiment, the members of the plurality can encode polypeptides that include different DNA contacting residues in the zinc finger domain(s). The polypeptides encoded by nucleic acids of the plurality can include other features described herein.

In another aspect, the invention features a library of polypeptides, the library including a plurality of polypeptides, each polypeptide of the plurality including (a) a zinc finger domain that varies from that other polypeptides of the plurality and (b) a protein transduction domain. For example, the plurality includes at least 10², 10⁴, 10⁶, or 10⁸ different members. The polypeptides of the plurality can include other features described herein. In one embodiment, the members of the plurality have different DNA contacting residues in the zinc finger domain. In a related aspect, the library includes a plurality of polypeptides, each polypeptide of the plurality including (a) a DNA binding domain that varies from that other polypeptides of the plurality and (b) a protein transduction domain.

The invention also features a method of selecting a zinc finger protein. The method can include: providing a library of polypeptides (e.g., transducible polypeptides as described herein); contacting a plurality of polypeptides from the library with one or more cells such that the polypeptides of the plurality enter the cells and the cells do not include a nucleic acid that encodes the polypeptide entering the respective cell; and evaluating a property of the one or more cells.

In one embodiment, each polypeptide of the plurality is contacted with a different cell, and the property of each of the different cells is evaluated. For example, the step of evaluating can include hybridization to a microarray, RT-PCR, a Northern analysis, or a Western analysis.

In another aspect, the invention features a method of evaluating a transducible DNA binding protein, the method including: providing a transducible DNA binding protein; administering the transducible DNA binding protein to a subject; and monitoring the subject. The subject can be monitored for a parameter affected by the transducible DNA binding protein. For example, the subject can be monitored by evaluating one or more cells, tissues, or sites in the subject. In one embodiment, the step of monitoring includes imaging the subject. For example, the transducible DNA binding protein is labeled, e.g., with a label that can be detected by non-invasive imaging, e.g., a MRI detectable label.

In another aspect, the invention features a method of altering (e.g., increasing or decreasing) gene expression in a eukaryotic cell. The method includes: contacting a eukaryotic cell with a chimeric DNA binding protein that comprises a zinc finger domain and a protein transduction domain. The protein is able to regulate transcription of an endogenous gene in the cell. The eukaryotic cell is typically a mammalian cell, e.g., a human, rodent, bovine, or canine cell. The cell can be a culture cell, e.g., maintained in tissue culture. In some cases, the cell is obtained from a subject or resides in a subject, e.g., a human subject. For example, the contacting can be performed in vitro or in vivo.

In one embodiment, the chimeric DNA binding protein comprises a plurality of zinc finger domains. The protein can include a plurality of zinc finger domains, e.g., two, three, four, five, or six zinc finger domains, or at least two, three, four, five, or six zinc finger domains. In one embodiment, the protein includes an array of zinc finger domains.

The protein can also include one or more of the following features: a nuclear localization signal, a dimerization domain, a cell targeting domain, cell surface protein binding domain, a purification handle and an effector domain. The protein can also include one or more non-peptide backbone bonds or an artificial amino acid. The cell targeting domain can include an immunoglobulin variable domain, a growth factor, a cell binding domain of a viral protein, or a cell binding domain of an extracellular protein.

In one embodiment, the protein transduction domain includes a viral sequence, e.g., from a virus that naturally infects humans, e.g., an HIV virus. An exemplary protein transduction domain is the HIV TAT protein transduction domain, e.g., the amino acid sequence: YGRKKRRQRRR (SEQ ID NO:1). The viral sequence can be between 5-50, or 8-20 amino acids in length. In another embodiment, the protein transduction domain includes a mammalian sequence, e.g., a sequence from a human protein. In another embodiment, the protein transduction domain includes an artificial sequence, e.g., a sequence identified as a transduction domain from a display library. For example, the protein transduction domain can include a modified tat protein transduction domain having the amino acid sequence of any one of SEQ ID NOs: 69 to 72 or a polyarginine oligopeptide consisting of 6 to 12 arginine residues.

The endogenous gene can be any endogenous gene, for example, jun B proto-oncogene, protein kinase C, lectin, brain-specific Na-dependent inorganic phosphate cotransporter, cellular retinoic acid-binding protein 1, cellular retinoic acid-binding protein 2, cadherin 13, H-cadherin (heart), vascular endothelial growth factor (VEGF-A), pigment epithelium-derived factor (PEDF), differentiation-related gene-1 (Drg-1), transcription factor E2F, early growth response-1 (EGR-1), protein tyrosine phosphatases 1B (PTP-1B), A20, Fas, melanoma differentiation associated gene-7 (MDA-7), presenilin-1 (PS-1), angiotensin converting enzyme, Angiopoietin-2, b-secretase (BACE1), mmp3, checkpoint with forkhead associated and ring finger (CHFR), peroxisome proliferator-activated receptor gamma (PPAR-gamma), TNF-related apoptosis-inducing ligand (TRAIL), Ku-80, ataxia-telangiectasia mutated (ATM), BRCA, CC-chemokine receptor 5 (CCR5), brain-derived neurotrophic factor (BDNF), tumor necrosis factor alpha-induced protein-3 (TNFAIP3) (A20), c-myc, Hypoxia-inducible factor-1 alpha (HIF-1 alpha), caspase-3, intercellular adhesion molecule type I (ICAM-1), angiotensin II receptor 1 (AT-1R), platelet-derived growth factor, insulin-like growth factor-I and -II, nerve growth factor, aFGF, bFGF, epidermal growth factor, TGF-α, TGF-β, erythropoietin, thrombopoietin, mucins, growth hormone, proinsulin, insulin A-chain, insulin B-chain, parathyroid hormone, thyroid stimulating hormone, thyroxine, follicle stimulating hormone, calcitonin, factor VIII, hematopoietic growth factor, enkephalinase, Mullerian-inhibiting substance, gonadotropin-associated peptide, tissue factor protein, inhibin, activin, interferon-α, interferon-β, interferon-γ, M-CSF, GM-CSF, G-CSF, IL-1, IL-2, IL-3, IL-4. IL-12, and IL-13.

In one embodiment, the chimeric DNA binding protein specifically binds to target gene. In another embodiment, the chimeric DNA binding protein specifically binds to a site within 1000, 500, 300, 100, 70, 50, 20, or 10 base pairs of the transcriptional start site, e.g., upstream or downstream. In another embodiment, the chimeric DNA binding protein specifically binds to a site within 1000, 500, 300, 100, 70, 50, 20, or 10 base pairs of the TATA box. In still other embodiments, the chimeric DNA binding protein specifically binds to a site within 100, 80, 70, 50, 30, 20, or 10 base pairs or a site that overlaps with a site bound by a naturally occurring transcription factor in a regulatory region of the endogenous gene. In one embodiment, the chimeric DNA binding protein increases expression of the endogenous gene, e.g., by at least 25, 50, 80, 100, 150, 200, or 500%. In another embodiment, the chimeric DNA binding protein decreases expression of the endogenous gene, e.g., to less than 80, 70, 60, 50, 40, 30, 20, 10, 5, or 2%.

In another embodiment, the step of monitoring includes determining a half life of the transducible DNA binding protein in the subject. In another embodiment, the step of monitoring includes determining a transcription profile for one or more cells in the subject. In another embodiment, the step of monitoring includes determining a profile of protein expression or modification states for one or more cells in the subject.

The term “dissociation constant” refers to the equilibrium dissociation constant (K_{d}) of a polypeptide for binding to a 28-basepair double-stranded DNA that includes one target site for the polypeptide being assayed. For example, if the polypeptide has a three finger DNA binding domain, the DNA will include a 9-bp or larger target site that the polypeptide specifically recognizes. The dissociation constant is determined by gel shift analysis using a purified protein that is bound in 20 mM Tris pH 7.7, 120 mM NaCl, 5 mM MgCl₂, 20 μM ZnSO₄, 10% glycerol, 0.1% Nonidet P-40, 5 mM DTT, and 0.10 mg/mL BSA (bovine serum albumin) at room temperature. Additional details are provided in the example below and Rebar and Pabo (1994) Science 263:671-673. Polypeptides that bind to sites larger than 28-basepairs can be assayed using a larger double-stranded DNA. Exemplary dissociation constants include constants less than 10⁻⁷ M, 10⁻⁸ M, 10⁻⁹ M, 10⁻¹⁰ M, 10⁻¹¹ M, or 10⁻¹² M.

The terms “hybrid” and “chimera” refer to a non-naturally occurring polypeptide that comprises amino acid sequences derived from either (i) at least two different naturally occurring sequences, or non-contiguous regions of the same naturally occurring sequence, wherein the non-contiguous regions are made contiguous in the hybrid; (ii) at least one artificial sequence (i.e., a sequence that does not occur naturally) and at least one naturally occurring sequence; or (iii) at least two artificial sequences (same or different).

When describing a sequence, the term “naturally occurring” refers to a sequence (e.g., a nucleic acid or amino acid sequence) which is present in a cell of a natural organism, i.e., an organism that has not been modified by molecular biological techniques. For example, a transgenic mouse is not a natural organism, but a highly inbred mouse that has not been modified by molecular biological techniques is considered natural. When describing a sequence, the term “viral” refers to a sequence of a naturally occurring virus, i.e., a virus that has not been modified by molecular biological techniques. One embodiment of the invention includes proteins that include a zinc finger domain from Homo sapiens, Mus musculus, Arabidopsis thaliana, Drosophila melanogaster, Escherichia coli, Saccharomyces cerevisiae, or Oryza saliva.

An “artificial sequence” is a sequence constructed by artificial means. Examples of artificial sequences include mutants of a naturally occurring sequence that are generated by site directed mutagenesis or random mutagenesis and de novo designed sequences.

The term “fusion” refers to a single polypeptide chain that includes the components that are fused. An exemplary fusion protein includes a DNA binding domain and a protein transduction domain. The fused components need not be directly linked. For example, another sequence (e.g., a linker or a functional domain) can be located between the fused elements.

The term “exogenous polypeptide” or “exogenous protein” refers to a polypeptide or protein that is introduced into a cell by artifice.

The term “isolated” describes a composition that is removed from at least 90% of at least one component of a sample (e.g., a natural sample or cell, e.g., a recombinant cell) or a synthetic reaction from which the isolated composition can be obtained. Compositions described herein produced artificially or naturally can be “compositions of at least” a certain degree of purity if the species or population of species of interest is at least 5, 10, 25, 50, 75, 80, 90, 95, 98, or 99% pure on a weight-weight basis, respectively.

The term “transducible” describes a compound that can cross a biological membrane and enter at least a mammalian cell. The terms “protein transduction domain” and “PTD” refer to an amino acid sequence that can cross a biological membrane, particularly a cell membrane. When attached to a heterologous polypeptide, a PTD can enhance the translocation of the heterologous polypeptide across a biological membrane. The term “PTD” does not refer to nuclear localization signals which facilitate transport of compounds through pores positioned in the nuclear envelope. Proteins entering the nucleus do not traverse an actual membrane bilayer.

The term “polypeptide” refers to a polymer of three or more amino acids linked by a peptide bond. The polypeptide may include one or more unnatural amino acids. Typically, the polypeptide includes only natural amino acids. The term “peptide” refers to a polypeptide that is between three and thirty-two amino acids in length. A “protein” can include one or more polypeptide chains. Accordingly, the term “protein” encompasses polypeptides and peptides. A protein or polypeptide can also include one or more modifications, e.g., a natural modification or an artificial modification. The term “domain” refers to a functional unit within a polypeptide. A domain's tertiary structure may be folded or unfolded.

The term “exogenous” refers to an agent that is supplied from without. An “endogenous gene” refers to any gene in a cell, including a viral gene which is introduced, e.g., by a virus, and a chromosomal gene, e.g., a naturally occurring chromosomal gene which is present in an unmodified cell. The methods and compositions described herein can be used to regulate naturally occurring endogenous genes, particularly those in unmodified cells. In addition methods for regulating endogenous genes can also be extended to regulate exogenous genes, e.g., genes introduced by artifice into a cell such as reporter genes and engineered recombinant nucleic acids.

The term “library” refers to a collection of different molecules. The library may be stored in a variety of forms. For example, each member of the collection may be present in a container with other members of the collection, e.g., all the other members of the collection. In another example, each member of the collection is isolated from other members of the collection. For example, library members can be arrayed or separately stored in wells or vials. A library can include a plurality of members that have a particular property. Such a library may also include other members, e.g., another plurality of members which does not have the particular property. It is also possible to store information about libraries, particular about members of the library, in a computer database.

All patents, patent applications, and references cited herein are incorporated by reference in their entirety. The following patent applications: WO 01/60970 (Kim et al.); U.S. Ser. No. 10/223,765, filed Aug. 19, 2002; U.S. Ser. No. 10/314,669, filed Dec. 9, 2002; U.S. Ser. No. 60/431,892, filed Dec. 9, 2002; and U.S. Ser. No. 60/453,111, filed Mar. 7, 2003, U.S. Ser. No. 60/477,459, are expressly incorporated by reference in their entirety for all purposes. The details of one or more embodiments of the invention are set forth in the accompanying drawings and the description below. Any feature described herein can be used in combination with another compatible feature also described herein. Other features, objects, and advantages of the invention will be apparent from the description and drawings, and from the claims.

### DESCRIPTION OF THE DRAWINGS

FIG. 1 depicts a gel of purified TAT-F435-KOX protein after Ni-NTA affinity chromatography.

FIG. 2 depicts a gel of purified TAT-F435-KOX protein after ion exchange chromatography.

FIG. 3 depicts the activation or repression of endogenous VEGF expression by TAT-F435-p65 or TAT-F435-KRAB.

FIG. 4 depicts the repression of VEGF-A protein production by TAT-F435-KOX in H460 cells.

FIG. 5 depicts the stability of Ni-NTA purified TAT-F435-KOX, wherein A represents the result of western blot employing proteins incubated in culture media; and B, the result of western blot employing proteins treated with protease inhibitor cocktail prior to incubation in culture media.

FIG. 6 depicts the result of western blot employing TAT-F435-KOX protein detected from H460 cells at various time points after transduction.

### DETAILED DESCRIPTION

The invention provides in part, artificial DNA binding proteins that can traverse biological membranes. In one implementation, these proteins can be used as therapeutic agents that are delivered to an extracellular milieu. The proteins then enter cells and cause a desired therapeutic effect. These transducible DNA binding proteins typically include a protein transduction domain (PTD).

This disclosure includes actual results that demonstrate that transducible DNA binding proteins are able to enter cells and regulate gene expression. Thus, transducible DNA binding proteins can be used as proteinaceous agents, particularly as proteinaceous drugs that are delivered to cells. These proteins can be delivered by any applicable route or by multiple routes. Thus, transducible DNA binding proteins, such as PTD-ZFP fusion proteins, can be used to treat diseases, disorders, and other conditions. In addition, these proteins can be used as research tools, both in vitro and in-vivo.

In one embodiment, the transducible DNA binding proteins include at least one zinc finger domain, e.g., a naturally occurring zinc finger domain. For example, artificial zinc finger proteins that include naturally occurring zinc finger domains can be designed to regulate the endogenous VEGF in a mammalian cell.

In one embodiment, the transducible DNA binding proteins are targeted to selected cells, tissues or organs.

Protein Transduction Domains

A “protein transduction domain” or “PTD” is an amino acid sequence that can cross a biological membrane, particularly a cell membrane. When attached to a heterologous polypeptide, a PTD can enhance the translocation of the heterologous polypeptide across a biological membrane. The PTD is typically covalently attached (e.g., by a peptide bond) to the heterologous DNA binding domain. For example, the PTD and the heterologous DNA binding domain can be encoded by a single nucleic acid, e.g., in a common open reading frame or in one or more exons of a common gene. An exemplary PTD can include between 10-30 amino acids and may form an amphipathic helix. Many PTD's are basic in character. For example, a basic PTD can include at least 4, 5, 6 or 8 basic residues (e.g., arginine or lysine). A PTD may be able to enhance the translocation of a polypeptide into a cell that lacks a cell wall or a cell from a particular species, e.g., a eukaryotic cell, e.g., a vertebrate cell, e.g., a mammalian cell, such as a human, simian, murine, bovine, equine, feline, or ovine cell.

A PTD can be linked to an artificial transcription factor, for example, using a flexible linker. Flexible linkers can include one or more glycine residues to allow for free rotation. For example, the PTD can be spaced from a DNA binding domain of the transcription factor by at least 10, 20, or 50 amino acids. A PTD can be located N- or C-terminal relative to a DNA binding domain. Being located N- or C-terminal to a particular domain does not require being adjacent to that particular domain. For example, a PTD N-terminal to a DNA binding domain can be separated from the DNA binding domain by a spacer and/or other types of domains.

A PTD can be chemically synthesized then conjugated chemically to separately prepared DNA binding domain with or without linker peptide.

An artificial transcription factor can also include a plurality of PTD's, e.g., a plurality of different PTD's or at least two copies of one PTD.

Exemplary PTD's include the following segments from the antennapedia protein, the herpes simplex virus VP22 protein and HIV TAT protein.

Tat. The Tat protein from human immunodeficiency virus type I (HIV-1) has the remarkable capacity to enter cells when added exogenously (Frankel A. D. and Pabo C. O. (1988) Cell 55:1189-1193, Mann D. A and Frankel A. D. (1991) EMBO J. 10:1733-1739, Fawell et al. (1994) Proc. Natl. Acad. Sci. USA 91:664-668). The minimal Tat PTD includes residues 47-57 of the human immunodeficiency virus Tat protein:

YGRKKRRQRRR (SEQ ID NO:1)

This peptide sequence is referred to as “TAT” herein. This peptide has been shown to successfully mediate the introduction of heterologous peptides and proteins in excess of 100 kDa into mammalian cells in vitro and in vivo (Ho et al. (2001) Cancer Res 61(2):474-7). Schwarze et al. showed that when the 120 kDa β-galactosidase protein fused with TAT was injected into mouse intraperitoneally, the fusion proteins were found in all types of cells and tissues even including brain, which has been thought to be difficult because of the blood-brain-barrier (Schwarze et al. (1999) Science 285(5433):1466-7).

Antennapedia. The antennapedia homeodomain also includes a peptide that is a PTD. Derossi et al. (1994) J. Bio. Chem. 269:10444-10450. This peptide, also referred to as “Penetratin”, includes the amino acid sequence:

AKIWFQNRRMKWKKENN (SEQ ID. NO:2)

VP22. The HSV VP22 protein also includes a PTD. This PTD is located at the VP22 C-terminal 34 amino acid residues:

DAATATRGRSAASRPTERPRAPARSASRPRRPVE (SEQ ID NO:3)

See, e.g., Elliott and O'Hare (1997) Cell 88:223-234 and U.S. Pat. No. 6,184,038.

Human PTD's. In one embodiment, the PTD is obtained from a human or other mammalian protein. Exemplary mammalian PTD's are described in WO 03/059940 (human SIM-2) and WO 03/059941 (Mph).

Cell-specific PTD's. Some PTD's are specific for particular cell types or states. One exemplary cell-specific PTD is the Hn1 synthetic peptide described in U.S. Published Application 2002-0102265. Hn1 is internalized by human head and neck squamous carcinoma cells and can be used to target an artificial transcription factor to a carcinoma, e.g., a carcinoma of the head or neck. The sequence of the HN1 synthetic peptide includes:

TSPLNIHNGQKL (SEQ ID NO:4)

or closely related sequences. U.S. Published Application 2002-0102265 also describes a general method for using phage display to identify other peptides and proteins which can function as cell specific PTD's. A phage display library that displays random peptides, such as the M13 phage peptide library PhD-12 from New England BioLabs (Beverly, Mass.), is incubated with target cells, e.g., cancer cells, in growth media. Internalized phages are recovered by lysing with TX-100 (1%) for 30 min at 37° C. and are amplified in a host E. coli strain. Although TX-100 does not lyse the nuclei, ionic detergents capable of disrupting nuclear membrane are avoided as they may inactivate the phage. Isolated phages are then counterselected against non-target cells, such as normal human fibroblasts. Phage that enter only target cells are sequenced and retested. See U.S. Pat. No. 6,451,527 for another exemplary method.

Synthetic PTD's. The minimal Tat PTD (aa 47-57) was modified to optimize protein transduction potential (Ho et al. (2001) Cancer Res 61(2):474-7). A FITC coupled with series of synthetic PTD's was tested with cultured T lymphocytes. Some synthetic PTD's showed enhanced protein transduction compared to Tat PTD. These PTD include;

YARKARRQARR (SEQ ID NO:69)
YARAARRAARR (SEQ ID NO:70)
YARAARRAARA (SEQ ID NO:71)
YARAAARQARA (SEQ ID NO:72)

Especially, the FITC conjugated with synthetic PID (YARAAARQARA; SEQ ID NO:72) showed enhanced uptake by whole blood cells when the mice were i.p. injected.

The poly-arginine peptides composed of about 6-12 arginine residues also can mediate protein transduction in some cases. For additional information about poly-arginine, see, e.g., Rothbard J B et al. Nat Med. 2000 6 (11):1253-7; Wender P A et al. Proc Natl Acad Sci USA. 2000 97(24):13003-8.

For additional information about PTD's, see also U.S. 2003-0082561; U.S. 2002-0102265; U.S. 2003-0040038; Schwarze et al. (1999) Science 285:1569-1572; Derossi et al. (1996) J. Biol. Chem. 271:18188; Hancock et al. (1991) EMBO J. 10:4033-4039; Buss et al. (1988) Mol. Cell. Biol. 8:3960-3963; Derossi et al. (1998) Trends in Cell Biology 8:84-87; Lindgren et al. (2000) Trends in Pharmacological Sciences 21:99-103; Kilic et al. (2003) Stroke 34:1304-10; Asoh et al. (2002) Proc Natl Acad Sci USA 99(26):17107-12; and Tanaka et al. (2003) J Immunol. 170(3):1291-8.

In addition to PTD's, cellular uptake signals can be used. Such signals include amino acid sequences which are specifically recognized by cellular receptors or other surface proteins. Interaction between the cellular uptake signal and the cell cause internalization of the artificial transcription factor that includes the cellular uptake signal. Some PTD's may also function by interaction with cellular receptors or other surface proteins.

Assays for protein transduction. A number of assays are available to determine if an amino acid sequence can function as a PTD. For example, the amino acid sequence can be fused to a reporter protein such as β-galactosidase to form a fusion protein. This fusion protein is contacted with culture cells. The cells are washed and then assayed for reporter activity. A specific implementation of this assay is described in Example 2 (1).

Another assay detects the presence of a fusion protein that includes the amino acid sequence in question and another detectable sequence, e.g., an epitope tag. This fusion protein is contacted with culture cells. The cells are washed and then analyzed by Western or immunofluorescence to detect presence of the detectable sequence in cells. A specific implementation of this assay is described in Example 2 (2).

Still other assays can be used to detect transcriptional regulatory activity of a fusion protein that includes the putative PTD the amino acid sequence in question, a DNA binding domain, and optionally an effector domain. For example, cells contacted with such fusion proteins can be assayed for the presence or level of mRNA or protein, e.g., using microarrays, mass spectroscopy, and high-throughput techniques.

Components of an Artificial Transcription Factor

An artificial transcription factor can include a DNA binding region that includes one or a plurality of DNA binding domains, e.g., a plurality of zinc finger domains. The transcription factor may also include a nuclear localization signal and an effector domain, e.g., a transcriptional regulatory domain.

DNA binding domains. A variety of protein structures are known to bind nucleic acids with high affinity and high specificity. These structures are used repeatedly in a myriad of different proteins to specifically control nucleic acid function (for reviews of structural motifs which recognize double stranded DNA, see, e.g., Pabo and Sauer (1992) Annu. Rev. Biochem. 61:1053-95; Patikoglou and Burley (1997) Annu. Rev. Biophys. Biomol. Struct. 26:289-325; Nelson (1995) Curr Opin GenetDev. 5:180-9). Examples of DNA binding domains include helix-loop-helix domains, helix-turn-helix domains, homeodomains, and zinc finger domains. Methods for identifying useful DNA binding domains are discussed below.

Zinc fingers. Zinc finger domains (or “ZFD's”) are small polypeptide domains of approximately 30 amino acid residues in which there are four residues, either cysteine or histidine, appropriately spaced such that they can coordinate a zinc ion (for reviews, see, e.g., Klug and Rhodes (1987) Trends Biochem. Sci. 12:464-469(1987); Evans and Hollenberg (1988) Cell 52:1-3; Payre and Vincent (1988) FEBS Lett. 234:245-250; Miller et al. (1985) EMBO J. 4:1609-1614; Berg (1988) Proc. Natl. Acad. Sci. U.S.A. 85:99-102; Rosenfeld and Margalit (1993) J. Biomol. Struct Dyn. 11:557-570). Hence, zinc finger domains can be categorized according to the identity of the residues that coordinate the zinc ion, e.g., as the Cys₂-His₂ class, the Cys₂-Cys₂ class, the Cys-CysHis class, and so forth. The zinc coordinating residues of Cys₂-His₂ zinc fingers are typically spaced as follows:

C-X₂₋₅-C-X₃-Xₐ-X₅-ψ-X₂-H-X₃₋₅-H, (SEQ ID NO:5)

where ψ (psi) is a hydrophobic residue (Wolfe et al. (1999) Annu. Rev. Biophys. Biomol. Struct. 3:183-212), “X” represents any amino acid, Xₐ is any amino acid (e.g., phenylalanine or tyrosine), the subscript number indicates the number of amino acids, and a subscript with two hyphenated numbers indicates a typical range of intervening amino acids. Typically, the intervening amino acids fold to form an anti-parallel β-sheet that packs against an α-helix, although the anti-parallel β-sheets can be short, non-ideal, or non-existent. The fold positions the zinc-coordinating side chains so they are in a tetrahedral conformation appropriate for coordinating the zinc ion. This fold positions the base contacting residues in conformation suitable for specifically recognizing basepairs in the DNA double helix.

For convenience, the primary DNA contacting residues of a zinc finger domain are numbered: −1, 2, 3, and 6 based on the following example:

(SEQ ID NO:6)

−1 1 2 3 4 5 6
C-X₂₋₅-C-X₃-Xₐ-X-R-X-D-E-X_{b}-X-R-H-X₃₋₅-H,

As noted in the example above, the DNA contacting residues are Arg (R), Asp (D), Glu (E), and Arg (R). The above motif can be abbreviated RDER. As used herein, such abbreviation is a shorthand that refers to a particular polypeptide sequence from the second residue preceding the first cysteine (above, initial residue of SEQ ID NO:6) to the ultimate metal-chelating histidine (ultimate residue of SEQ ID NO:6). In the above motif and others, Xₐ is frequently aromatic, and X_{b} is frequently hydrophobic. Where two different sequences have the same motif, a number may be used to indicate each sequence (e.g., RDER1 or RDER2).

In certain contexts where made explicitly apparent, the four-letter abbreviation refers to the motif in general. In other words, the motif specifies the amino acids at positions −1, 2, 3, and 6, while the other positions can be any amino acid, typically, but not necessarily, a non-cysteine amino acid. The small letter “m” before a motif can be used to make explicit that the abbreviation is referring to a motif. For example, mRDER refers to a motif in which R appears at positions −1, D at position 2, E at position 3, and R at position 6.

A zinc finger DNA-binding protein may include two or more zinc finger domains, typically at least three zinc finger domains. For example, the protein can include at least four, five, six, eight, twelve or more zinc finger domains. In one embodiment, the zinc finger domains are located in a tandem array, e.g., of three or more zinc finger domains. A tandem array includes domains that are within ten amino acids of each other and that are not separated by other types of functional domains.

Zinc finger domains are present in species from yeast to higher plants and to humans. By one estimate, there are at least several thousand zinc finger domains in the human genome alone, possibly at least 4,500. Naturally occurring zinc finger domains can be identified in or isolated from zinc finger proteins. Non-limiting examples of zinc finger proteins include CF2-II; Kruppel; WT1; basonuclin; BCL-6/LAZ-3; erythroid Kruppel-like transcription factor; transcription factors Sp1, Sp2, Sp3, and Sp4; transcriptional repressor YY1; EGR1/Krox24; EGR2/Krox20; EGR3/Pilot; EGR4/AT133; Evi-1; GLI1; GLI2; GLI3; HIV-EP1/ZNF40; HIV-EP2; KR1; ZfX; ZfY; and ZNF7.

Activation domains. One type of effector domain is a transcriptional activation domain. Transcriptional activation domains increase the amount of transcription of a gene when recruited to a regulatory region of the gene. Exemplary activation domains include the Gal4 activation domain from yeast and the VP16 domain from herpes simplex virus. The ability of a domain to activate transcription can be determined by fusing the domain to a known DNA binding domain and then determining if a reporter gene operably linked to a site recognized by the known DNA-binding domain is activated by the fusion protein.

An exemplary activation domain is the following domain from p65:

(SEQ ID NO:7)

YLPDTDDRHRIEEKRKRTYETFKSIMKKSPFSGPTDPRPPPRRIAVPSRS
SASVPKPAPQPYPFTSSLSTINYDEFPTMVFPSGQISQASALAPAPPQVL
PQAPAPAPAPAMVSALAQAPAPVPVLAPGPPQAVAPPAPKPTQAGEGTLS
EALLQLQFDDEDLGALLGNSTDPAVFTDLASVDNSEFQQLLNQGIPVAPH
TTEPMLMEYPEAITRLVTAQRPPDPAPAPLGAPGLPNGLLSGDEDFSSIA
DMDFSALLSQ

The sequence of an exemplary Gal4 activation domain is as follows:

(SEQ ID NO:8)

NFNQSGNIADSSLSFTFTNSSNGPNLITTQTNSQALSQPIASSNVHDNFM
NNEITASKIDDGNNSKPLSPGWTDQTAYNAFGITTGMFNTTTMDDVYNYL
FDDEDTPPNPKKEISMAYPYDVPDYAS

In bacteria, activation domain function can be emulated by a domain that recruits a wild-type RNA polymerase alpha subunit C-terminal domain or a mutant alpha subunit C-terminal domain, e.g., a C-terminal domain fused to a protein interaction domain.

Repression domains. If desired, a repression domain (e.g., instead of an activation domain) can be fused to the DNA binding domain. Examples of eukaryotic repression domains include repression domains from Kid, UME6, ORANGE, groucho, and WRPW (see, e.g., Dawson et al. (1995) Mol. Cell Biol. 15:6923-31). The ability of a domain to repress transcription can be confirmed by fusing the domain to a known DNA binding domain and then determining if a reporter gene operably linked to sites recognized by the known DNA-binding domain is repressed by the fusion protein.

An exemplary repression domain is the following domain from UM6 protein:

(SEQ ID NO:9)

NSASSSTKLDDDLGTAAAVLSNMRSSPYRTHDKPISNVNDMNNTNALGVP
ASRPHSSSFPSKGVLRPILLRIHNSEQQPIFESNNSTACI

Another exemplary repression domain is from the Kid protein:

(SEQ ID NO:10)

VSVTFEDVAVLFTRDEWKKLDLSQRSLYREVMLENYSNLASMAGFLFTKP
KVISLLQQGEDPW

KOX repression domain: This domain includes the “KRAB” domain from the human Kox1 protein (Zinc finger protein 10; NCBI protein database AAH24182; GI:18848329), i.e., amino acids 2-97 of Kox1:

(SEQ ID NO:11)

DAIKLTAWSRTLVTFKDVFVDFTREEWKLLDTAQQIVYRNVMLENYKNLV
SLGYQLTKPDVILRLEKGEEPWLVEREIHQETHPDSETAFEIKSSV

Still other chimeric transcription factors include neither an activation or repression domain. Rather, such transcription factors may alter transcription by displacing or otherwise competing with a bound endogenous transcription factor (e.g., an activator or repressor).

Other types of effector domains include domains that associated with one or more of the following activitie's: histone modification (e.g., acetylation, deacetylation, ubiquitination), chromatin structure packaging, DNA cleavage, topoisomerase activity, and DNA methylation state (e.g., methylation or demethylation).

Additional Features for Designed Transcription Factors

Linkers. DNA binding domains can be connected by a variety of linkers. The utility and design of linkers are well known in the art. A particularly useful linker is a peptide linker that is encoded by nucleic acid. Thus, one can construct a synthetic gene that encodes a first DNA binding domain, the peptide linker, and a second DNA binding domain. This design can be repeated in order to construct large, synthetic, multi-domain DNA binding proteins. PCT WO 99/45132 and Kim et al. ((1998) Proc. Natl. Acad. Sci. USA 95:2812-7) describe the design of peptide linkers suitable for joining zinc finger domains. For implementations utilizing zinc finger domains, a peptide that occurs naturally between zinc fingers can be used as a linker to join fingers together. A typical such naturally occurring linker is: Thr-Gly-(Glu or Gln)-(Lys or Arg)-Pro-(Tyr or Phe).

Additional peptide linkers are available that form random coil, α-helical or β-pleated tertiary structures. Polypeptides that form suitable flexible liners are well known in the art (see, e.g., Robinson et al. (1998) Proc Natl Acad Sci USA. 95:5929-34). Flexible linkers typically include glycine, because this amino acid, which lacks a side chain, is unique in its rotational freedom. Serine or threonine can be interspersed in the linker to increase hydrophilicity. In additional, amino acids capable of interacting with the phosphate backbone of DNA can be utilized in order to increase binding affinity. Judicious use of such amino acids allows for balancing increases in affinity with loss of sequence specificity. If a rigid extension is desirable as a linker, α-helical linkers, such as the helical linker described in Pantoliano et al. (1991) Biochem. 30:10117-10125, can be used. Linkers can also be designed by computer modeling (see, e.g., U.S. Pat. No. 4,946,778). Software for molecular modeling is commercially available (e.g., from Molecular Simulations, Inc., San Diego, Calif.). The linker is optionally optimized, e.g., to reduce antigenicity and/or to increase stability, using standard mutagenesis techniques and appropriate biophysical tests as practiced in the art of protein engineering, and functional assays as described herein. As mentioned above, flexible linkers can also be used to connect a PTD to a DNA binding domain or to another domain of an artificial DNA binding protein.

An alternative to a peptide linker is a linker which uses other types of chemical bonds. Accordingly, a PTD and a DNA binding domain can be linked by a synthetic, non-peptidyl linker. A polypeptide that includes a PTD can be coupled to a polypeptide that includes a DNA binding domain in a synthetic reaction. Homo- or heterobifunctional crosslinkers can be used. In one embodiment, the synthetic linker is cleaved in a cell. For example, the synthetic linker can include a reducible thiol bond. Examples of synthetic linkers include: BM[PEO]₃ (1,8-bis-Maleimidotriethyleneglycol, OCOES (Bis[2-(succinimidooxycarbonyloxy)ethyl]sulfone), and DSG (Disuccinimidyl glutarate).

Dimerization Domains. An alternative method of linking DNA binding domains is the use of dimerization domains, especially heterodimerization domains (see, e.g., Pomerantz et al. (1998) Biochemistry 37:965-970). In this implementation, DNA binding domains are present in separate polypeptide chains. For example, a first polypeptide encodes DNA binding domain A, linker, and domain B, while a second polypeptide encodes domain C, linker, and domain D. One or both of these polypeptides can also include a PTD.

An artisan can select a dimerization domain from the many well-characterized dimerization domains. Domains that favor heterodimerization can be used if homodimers are not desired. A particularly adaptable dimerization domain is the coiled-coil motif, e.g., a dimeric parallel or anti-parallel coiled-coil. Coiled-coil sequences that preferentially form heterodimers are also available (Lumb et al. (1995) Biochemistry 34:8642-8648). Another species of dimerization domain is one in which dimerization is triggered by a small molecule or by a signaling event. For example, a dimeric form of FK506 can be used to dimerize two FK506 binding protein (FKBP) domains. Such dimerization domains can be utilized to provide additional levels of regulation.

Dimerization can be stabilized by a disulfide bond if cysteines are engineered at opposing positions on the dimerization interface. Where DNA binding domains are linked by a disulfide, a PTD on one polypeptide can be used to transduce the partner polypeptide from the extracellular environment into a cell. Once in the cell, the disulfide bond may be reduced and the dimerization may be stabilized by other interactions, e.g., non-covalent interactions.

Design of Novel DNA-Binding Proteins

An artificial DNA-binding protein can be rationally constructed to recognize a target sequence by mixing and matching characterized zinc finger domains. Zinc finger domains can be isolated and characterized using a variety of methods. One known method for constructing an artificial DNA-binding protein includes using phage display to select for zinc finger domains with altered DNA-binding specificity (Greisman and Pabo (1997) Science 275:657-61). Domains that interact with a target sequence are selected and used to generate a DNA binding protein that binds to the target sequence.

Bae K H et al. (2003) Nat Biotechnol. 21(3):275-80 describes a method for evaluating the specificity of DNA-binding domains in cells and a method of constructing new DNA-binding proteins using information from such cellular assays. WO 01/60970 and WO 03/016571 also describes methods for designing DNA-binding proteins. The modular structure of zinc finger domains facilitates their rearrangement to construct new DNA-binding proteins. Zinc finger domains in the naturally-occurring Zif268 protein are positioned in a tandem array that can straddle the DNA double helix. Each domain independently recognizes a different 3-4 basepair DNA segment. By linking three or more zinc finger domains, a DNA binding protein that specifically recognizes a 9-bp or longer DNA sequence can be engineered.

A Database of Zinc Finger Domains. The one-hybrid selection system described in WO 01/60970 can be utilized to identify one or more zinc finger domains for each possible 3- or 4-basepair binding site or a representative number of such binding sites. The results of this process can be accumulated as a series of associations between a zinc finger domain and its preferred 3- or 4-basepair binding site or sites. Examples of such associations are provided in Table 1.

The results can also be stored in a machine as a database, e.g., a relational database, spreadsheet, or text file. Each record of such a database associates a representation of a zinc finger domain and a string indicating the sequence of the one or more preferred binding sites of the domain. The database record can include an indication of the relative affinity of the zinc finger domains that bind each site. In some implementations, the database record can also include information that indicates the physical location of the nucleic acid encoding the particular zinc finger domain. Such a physical location can be, for example, a particular well of a microtitre plate stored in a freezer.

The database can be configured so that it can be queried or filtered, e.g., using a SQL operating environment, a scripting language (such as PERL or a Microsoft Excel® macro), or a programming language. Such a database would enable a user to identify one or more zinc finger domains that recognize a particular 3- or 4-basepair binding site. Database and other information such as can be stored on a database server can also be configured to communicate with each device using commands and other signals that are interpretable by the device. The computer-based aspects of the system can be implemented in digital electronic circuitry, or in computer hardware, firmware, software, or in combinations thereof. An apparatus of the invention, e.g., the database server, can be implemented in a computer program product tangibly embodied in a machine-readable storage device for execution by a programmable processor; and method actions can be performed by a programmable processor executing a program of instructions to perform functions of the invention by operating on input data and generating output. One non-limiting example of an execution environment includes computers running Windows XP or Windows NT 4.0 (Microsoft) or better or Solaris 2.6 or better (Sun Microsystems) operating systems.

The zinc finger domains can also be tested in the context of multiple different fusion proteins to verify their specificity. Moreover, particular binding sites for which a paucity of domains is available can be the target of additional selection screens. Libraries for such selections can be prepared by mutagenizing a zinc finger domain that binds a similar yet distinct site. A complete matrix of zinc finger domains for each possible binding site is not essential, as the domains can be staggered relative to the target binding site in order to best utilize the domains available. Such staggering can be accomplished both by parsing the binding site in the most useful 3 or 4 basepair binding sites, and also by varying the linker length between zinc finger domains. In order to incorporate both selectivity and high affinity into the design polypeptide, zinc finger domains that have high specificity for a desired site can be flanked by other domains that bind with higher affinity, but lesser specificity. The in vivo screening method described herein can used to test the in vivo function, affnity, and specificity of an artificially assembled zinc finger protein and derivatives thereof. Likewise, the method can be used to optimize such assembled proteins, e.g., by creating libraries of varied linker composition, zinc finger domain modules, zinc finger domain compositions, and so forth.

Parsing a target site. The target 9-bp or longer DNA sequence is parsed into 3- or 4-bp segments. Zinc finger domains are identified (e.g., from a database described above) that recognize each parsed 3- or 4-bp segment. Longer target sequences, e.g., 20 bp to 500 bp sequences, are also suitable targets as 9 bp, 12 bp, and 15 bp subsequences can be identified within them. In particular, subsequences amenable for parsing into sites well represented in the database can serve as initial design targets.

A scoring regime can be used to estimate the probability that a particular designed chimeric zinc finger protein would recognize the target site in the cell. The scores can be a function of each component finger's affinity for its preferred subsites, its specificity, and its success in previously designed proteins.

Computer Programs. Computer systems and software can be used to access a machine-readable database described above, parse a target site, and output one or more chimeric zinc finger protein designs.

The techniques may be implemented in programs executing on programmable machines such as mobile or stationary computers, and similar devices that each include a processor, a storage medium readable by the processor, and one or more output devices. Each program may be implemented in a high level procedural or object oriented programming language to communicate with a machine system. Some merely illustrative examples of computer languages include C, C++, Java, Fortran, and Visual Basic.

Each such program may be stored on a storage medium or device, e.g., compact disc read only memory (CD-ROM), hard disk, magnetic diskette, or similar medium or device, that is readable by a general or special purpose programmable machine for configuring and operating the machine when the storage medium or device is read by the computer to perform the procedures described in this document. The system may also be implemented as a machine-readable storage medium, configured with a program, where the storage medium so configured causes a machine to operate in a specific and predefined manner.

The computer system can be connected to an internal or external network. For example, the computer system can receive requests from a remotely located client system, e.g., using HTTP, HTTPS, or XML protocols. The requests can be an identifier for a known target gene or a string representing the sequence of a target nucleic acid. In the former case, the computer system can access a sequence database such as GenBank to retrieve the nucleic acid sequence of regulatory regions of the target gene. The sequence of the regulatory region or the directly-received target nucleic acid sequence is then parsed into subsites, and chimeric zinc finger proteins are designed, e.g., as described above.

The system can communicate the results to the remotely located client. Alternatively, the system can control a robot to physically retrieve nucleic acids encoding the designed chimeric zinc finger proteins. In this implementation, a library of nucleic acids encoding chimeric zinc finger proteins is constructed and stored, e.g., as frozen purified DNA or frozen bacterial strains harboring the nucleic acids. The robot responds to signals from the computer system by accessing specified addresses of the library. The retrieved nucleic acids can then be processed, packaged and delivered to the client. Alternatively, the retrieved nucleic acids can be introduced into cells and assayed. The computer system can then communicate the results of the assay to the client across the network.

Constructing a Protein from Selected Modules. Once a chimeric polypeptide sequence containing multiple zinc finger domains is designed, a nucleic acid sequence encoding the designed polypeptide sequence can be synthesized. Methods for constructing synthetic genes are routine in the art. Such methods include gene construction from custom synthesized oligonucleotides, PCR mediated cloning, and mega-primer PCR. Additional sequences can be joined to the nucleic acid encoding the designed polypeptide sequence. The additional sequence can provide regulatory functions or a sequence coding for an amino acid sequence with a desired function. Examples of such additional sequences are described herein.

Libraries of PTD-fusions. It is also possible to evaluate a plurality of combinations of zinc finger domains by producing a library of polypeptides that includes a plurality of polypeptides that each includes a zinc finger domain and a PTD. The library can be screened by evaluating the ability of each protein of the plurality to alter a parameter of a cell, e.g., expression of a gene or a discernable phenotype.

Phenotypic Screening or Selection

It is also possible to screen libraries of nucleic acids encoding different combinations of zinc finger domains to identify a polypeptide that includes a functional DNA binding domain that produces a desired phenotypic effect. U.S. Ser. No. 10/314,669, filed Dec. 9, 2002, describes exemplary methods of identifying useful zinc finger proteins by screening or selection. Generally, a library of nucleic acid that encodes polypeptides that include different combination of zinc finger domains and an effector domain is prepared and introduced into cells. After expressing the library members, cells that exhibit an altered phenotype relative to a reference cell (e.g., an untransformed cell or a cell transformed with a vector nucleic acid) are isolated. The library nucleic acid in the cell is recovered and characterized. The nucleic acid can then be modified to produce a nucleic acid that encodes a polypeptide that includes the DNA binding domains and a PTD.

Exemplary Zinc Finger Domains

An artificial transcription factor can include chimeras of zinc finger domains. In one embodiment, one or more of the zinc finger domains is naturally occurring. Many exemplary human zinc finger domains are described in WO 01/60970, WO 03/016571, and U.S. Ser. No. 10/223,765. See also Table 1 below. The binding specificities of each domain, listed in the last column, can be used to design a transcription factor with a particular specificity.

TABLE 1

| | | | |
|---|---|---|---|
| | | SEQ ID | |
| ZFD | Amino acid sequence | NO: | Target subsite (s) |

| | | | |
|---|---|---|---|
| CSNR1 | YKCKQCGKAFGCPSNLRRHGRTH | 12 | GAA > GAC > GAG |
| CSNR2 | YQCNICGKCFSCNSNLHRHQRTH | 13 | GAA > GAC > GAG |
| DSAR | YSCGICGKSFSDSSAKRRHCILH | 14 | GTC |
| DSCR | YTCSDCGKAFRDKSCLNRHRRTH | 15 | GCC |
| HSNK | YKCKECGKAFNHSSNFNKHHRIH | 16 | GAC |
| HSSR | FKCPVCGKAFRHSSSLVRHQRTH | 17 | GTT |
| ISNR | YRCKYCDRSFSISSNLQRHVRNIH | 18 | GAA > GAT > G.AC |
| ISNV | YECDHCGKAFSIGSNLNVHRRIH | 19 | AAT |
| KSNR | YGCHLCGKAFSKSSNLRRHEMIH | 20 | GAG |
| QAHR | YKCKECGQAFRQRAHLIRHHKLH | 21 | GGA |
| QFNR | YKCHQCGKAFIQSFNLRRHERTH | 22 | GAG |
| QGNR | FQCNQCGASFTQKGNLLRHIKLH | 23 | GAA |
| QSHR1 | YACHLCGKAFTQSSHLRRHEKTH | 24 | GGA > GAA > AGA |
| QSHR2 | YKCGQCGKFYSQVSHLTRHQKIH | 25 | GGA |
| QSHR3 | YACHLCGKAFTQCSHLRRHEKTH | 26 | GGA > GAA |
| QSHR4 | YACHLCAKAFIQCSHLRRHEKTH | 27 | GGA > GAA |
| QSHR5 | YVCRECGRGFRQHSHLVRHKRTH | 28 | GGA > AGA > GAA > CGA |
| QSHT | YKCEECGKAFRQSSHLTTHKIIH | 29 | AGA, CGA > TGA > GGA |
| QSHV | YECDHCGKSFSQSSHLNVHKRTH | 30 | CGA > AGA > TGA |
| QSNI | YMCSECGRGFSQKSNLIIHQRTH | 31 | AAA, CAA |
| QSNK | YKCEECGKAFTQSSNLTKHKKIH | 32 | GAA > TAA > AAA |
| QSNR1 | FECKDCGKAFIQKSNLIRHQRTH | 33 | GAA |
| QSNR2 | YVCRECRRGFSQKSNLIRHQRTH | 34 | GAA |
| QSNR3 | YECEKCGKAFNQSSNLTRHKKSH | 35 | GAA |
| QSNV1 | YECNTCRKTFSQKSNLIVHQRTH | 36 | AAA > CAA |
| QSNV2 | YVCSKCGKAFTQSSNLTVHQKIH | 37 | AAA > CAA |
| QSNV3 | YKCDECGKNFTQSSNLIVHKRIH | 38 | AAA |
| QSNV4 | YECDVCGKTFTQKSNLGVHQRTH | 39 | AAA |
| QSNT | YECVQCGKGFTQSSNLITHQRVH | 40 | AAA |
| QSSR1 | YKCPDCGKSFSQSSSLIRHQRTH | 41 | GTA > GCA |
| QSSR2 | YECQDCGRAFNQNSSLGRHKRTH | 42 | GTA |
| QSSR3 | YECNECGKFFSQSSSLIRHRRSH | 43 | GTA > GCA |
| QSTR | YKCEECGKAFNQSSTLTRHKIVH | 44 | GTA > GCA |
| QSTV | YECNECGKAFAQNSTLRVHQRIH | 45 | ACA |
| QTHQ | YECHDCGKSFRQSTHLTQHRRIH | 46 | AGA > CGA, TGA |
| QTHR1 | YECHDCGKSFRQSTHLTRHRRIH | 47 | GGA > AGA, GAA |
| QTHR2 | HKCLECGKCFSQNTHLTRHQRTH | 48 | GGA |
| RDER1 | YVCDVEGCTWKFARSDELNRHKKRH | 49 | GCG > GTG, GAC |
| RDER2 | YHCDWDGCGWKFARSDELTRHYRKH | 50 | GCG > GTG |
| RDER3 | YRCSWEGCEWRFARSDELTRHFRKH | 51 | GCG > GTG |
| RDER4 | FSCSWKGCERRFARSDELSRHRRTH | 52 | GCG > GTG |
| RDER5 | FACSWQDCNKKFARSDELARHYRTH | 53 | GCG |
| RDER6 | YHCNWDGCGWKFARSDELTRHYRKH | 54 | GCG > GTG |
| RDHR1 | FLCQYCAQRFGRKDHLTRHMKKSH | 55 | GAG, GGG |
| RDHT | FQCKTCQRKFSRSDHLKTHTRTH | 56 | AGG, CGG, GGG, TGG |
| RDKI | FACEVCGVRFTRNDKLKIHMRKH | 57 | GGG |
| RDKR | YVCDVEGCTWKFARSDKLNRHKKRH | 58 | GGG > AGG |
| RSHR | YKCMECGKAFNRRSHLTRHQRIH | 59 | GGG |
| RSNR | YICRKCGRGFSRKSNLIRHQRTH | 60 | GAG > GTG |
| RTNR | YLCSECDKCFSRSTNLIRHRRTH | 61 | GAG |
| SSNR | YECKECGKAFSSGSNFTRHQRIH | 62 | GAG > GAC |
| VSNV | YECDHCGKAFSVSSNLNVHRRIH | 63 | AAT > CAT > TAT |
| VSSR | YTCKQCGKAFSVSSSLRRHETTH | 64 | GTT > GTG > GTA |
| VSTR | YECNYCGKTFSVSSTLIRHQRIH | 65 | GCT > GCG |
| WSNR | YRCEECGKAFRWPSNLTRHKRIH | 66 | GGT > GGA |

Particular combinations of zinc finger domains that produce useful DNA binding domains have been described. See, e.g., WO 01/60970, WO 03/016571, U.S. Ser. No. 10/314,669 and U.S. Ser. No. 60/431,892.

U.S. Ser. Nos. 60/431,892 and 10/732,620 describes, inter alia, DNA binding domains that can bind to regulatory sequences of the VEGF gene and regulate VEGF gene transcription. F121 and F475 are two exemplary DNA binding domains that bind to regulatory sequences of the VEGF gene.

An exemplary F475 protein can include the following amino acid sequence:

(SEQ ID NO:67)

YKCGQCGKFYSQVSHLTRHQKIHTGEKPFQCKTCQRKFSRSDHLKTHTRT
HTGEKPYICRKCGRGFSRKSNLIRHQRTHTGEK

An exemplary F121 protein can include the following amino acid sequence:

(SEQ ID NO:68)

YKCEECGKAFRQSSHLTTHKIIHTGEKPYKCMECGKAFNRRSHLTRHQRI
HTGEKPFQCKTCQRKFSRSDHLKTHTRTHTGEK

A DNA binding domain that includes zinc finger domains having at least two zinc finger domains (e.g., two or three domains, in the same respective order) that have DNA contacting residues identical to those of the zinc finger domains in F475 and F121 can also be used.

Further examples of zinc finger proteins include those described in Table 2 below:

TABLE 2

| Name | Motifs (Col. 2) | Specific Domains (Col. 3) |
|---|---|---|
| F475 | MQSHR-mRDHT-mRSNR | QSHR2-RDHT-RSNR |
| F121 | MQSHT-mRSHR-mRDHT | QSHT-RSHR-RDHT |
| F435 | MQSHR-mRDHT-mRSHR | QSHR2-RDHT-RSHR |
| F547 | MRSHR-mRDHT-mVSNV | RSHR-RDHT-VSNV |
| F2825 | MQSHV-mRDHR-mRDHT | QSHV-RDHR1-RDHT |
| F480 | MRSHR-mRDHT-mRSHR | RSHR-RDHT-RSHR |
| F2828 | MCSNR-mWSNR-mRDHR | CSNR1-WSNR-RDHR1 |
| F625 | MCSNR-mWSNR-mRSHR | CSNR1-WSNR-RSHR |
| F2830 | MDSNR-mWSNR-mRDHR | DSNRa-WSNR-RDHR1 |
| F2838 | MDSNR-mWSNR-mRSHR | DSNRa-WSNR-RSHR |
| F109 | MRDER-mQSSR-mQSHT-mRSNR | RDER1-QSSR1-QSHT-RSNR |
| F2604 | MDSAR-mRSNR-mRDHT-mVSSR | DSAR2-RSNR-RDHT-VSSR |
| F2605 | MQSHT-mDSAR-mRSNR-mRDHT | QSHT-DSAR2-RSNR-RDHT |
| F2607 | MRDHT-mVSNV-mQSHT-mDSAR | RDHT-VSNV-QSHT-DSAR2 |
| F2615 | MRSHR-mDSCR-mQSHT-mDSCR | RSHR-DSCR-QSHT-DSCR |
| F2633 | MQSNR-mQSHR-mRDHT-mRSNR | QSNR3-QSHR2-RDHT-RSNR |
| F2634 | MCSNR-mRDHT-mRSNR-mRSHR | CSNR1-RDHT-RSNR-RSHR |
| F2636 | MRSHR-mQSHT-mRSHR-mRDER | RSHR-QSHT-RSHR-RDER1 |
| F2644 | MQSNR-mRSHR-mQSSR-mRSHR | QSNR3-RSHR-QSSR1-RSHR |
| F2646 | MQSHT-mDSCR-mRDHT-mCSNR | QSHT-DSCR-RDHT-CSNR1 |
| F2650 | MQSHT-mWSNR-mRSHR-mWSNR | QSHT-WSNR-RSHR-WSNR |
| F2679 | MVSNV-mRSHR-mRDER-mQSNV | VSNV-RSHR-RDER1-QSNV2 |
| F2610 | MRSNR-mRSHR-mRDHT-mRSHR | RSNR-RSHR-RDHT-RSHR |
| F2612 | MRSHR-mRDHT-mRSHR-mRDHT | RSHR-RDHT-RSHR-RDHT |
| F2638 | MRSNR-mQSHR-mRDHT-mRSHR | RSNR-QSHR2-RDHT-RSHR |
| F2608 | MRSHR-mRDHT-mVSNV-mQSHT | RSHR-RDHT-VSNV-QSHT |
| F2611 | MRSHR-mRSHR-mWSNR-mRSHR | RSHR-RSHR-WSNR-RSHR |
| F2617 | MRDER-mRSHR-mDSCR-mQSHT | RDER1-RSHR-DSCR-QSHT |
| F2619 | MRSHR-mVSTR-mQSNR-mRDHT | RSHR-VSTR-QSNR3-RDHT |
| F2623 | MQSHT-mRSNR-mWSNR-mRDER | QSHT-RSNR-WSNR-RDER1 |
| F2625 | MQSHT-mWSNR-mRDHT-mRDER | QSHT-WSNR-RDHT-RDER1 |
| F2628 | MVSSR-mWSNR-mRSNR-mVSSR | VSSR-WSNR-RSNR-VSSR |
| F2629 | MQSHR-mVSSR-mWSNR-mRSNR | QSHR2-VSSR-WSNR-RSNR |
| F2630 | MRDER-mQSHR-mVSSR-mWSNR | RDER1-QSHR2-VSSR-WSNR |
| F2635 | MQSHR-mRSNR-mQSHR-mRDHT | QSHR2-RSNR-QSHR2-RDHT |
| F2637 | MRDHT-mRSNR-mRSHR-mWSNR | RDHT-RSNR-RSHR-WSNR |
| F2642 | MRDHT-mRSHR-mCSNR-mRDHT | RDHT-RSHR-CSNR1-RDHT |
| F2643 | MRSHR-mCSNR-mRDHT-mCSNR | RSHR-CSNR1-RDHT-CSNR1 |
| F2648 | MQSSR-mQSHR-mRSNR-mRSNR | QSSR1-QSHR2-RSNR-RSNR |
| F2651 | MVSTR-mQSHT-mWSNR-mRSHR | VSTR-QSHT-WSNR-RSHR |
| F2653 | MVSTR-mQSNR-mRSHR-mQSNR | VSTR-QSNR3-RSHR-QSNR3 |
| F2654 | MQSNR-mRSHR-mQSNR-mVSNV | QSNR3-RSHR-QSNR3-VSNV |
| F2662 | MDSCR-mRDHT-mVSTR-mRDER | DSCR-RDHT-VSTR-RDER1 |
| F2667 | MRSHR-mDSCR-mRDHT-mRSHR | RSHR-DSCR-RDHT-RSHR |
| F2668 | MRSHR-mRSHR-mQSNV-mQSNV | RSHR-RSHR-QSNV2-QSNV2 |
| F2673 | MRDHT-mVSSR-mRDER-mQSSR | RDHT-VSSR-RDER1-QSSR1 |
| F2682 | MRSNR-mQSSR-mQSNR-mRSHR | RSNR-QSSR1-QSNR3-RSHR |
| F2689 | MRSNR-mDSAR-mQSNR-mQSHT | RSNR-DSAR2-QSNR3-QSHT |
| F2697 | MRSHR-mCSNR-mQSHT-mRSNR | RSHR-CSNR1-QSHT-RSNR |
| F2699 | MRSNR-mQSHT-mDSAR-mRSHR | RSNR-QSHT-DSAR2-RSHR |
| F2703 | MQSHR-mRSHR-mRDER-mRSHR | QSHR2-RSHR-RDER1-RSHR |
| F2702 | MRSHR-mQSHR-mRSHR-mQSNV | RSHR-QSHR2-RSHR-QSNV2 |

Preferred zinc finger proteins among those described in Table 2 are F475, F121, F435, F547, F2825, F109, F2604, F2605, F2607, F2615, F2633, F2634, F2636, F2644, F2646, F2650 and F2679.

Such exemplary proteins include those that include at least two, three or four of the specific domains in column 3 or those that include zinc finger domains that have at least two, three or four of the same motifs as those in column 2. Still other examples are proteins that compete with the above proteins for binding to a target site, e.g., in the VEGF-A gene.

U.S. Ser. No. 10/314,669 describes, inter alia, DNA binding domains that can regulate (1) production of a secreted protein (e.g., insulin), (2) stress resistance, (3) differentiation state (e.g., neuronal or oosteogenic differentiation), and (4) proliferation.

Functional Assays and Uses

The function of a transducible DNA binding protein can be assayed in vitro or in vivo. Exemplary functional assays for an artificial transcription factor include an in vitro binding assay, SELEX, in vivo reporter gene regulation, and transcriptional profiling.

Assaying binding site preferences. The binding site preference of each domain can be verified by a biochemical assay such as EMSA, DNase footprinting, surface plasmon resonance, SELEX, or column binding. The substrate for binding can be a synthetic oligonucleotide encompassing the target site. The assay can also include non-specific DNA as a competitor, or specific DNA sequences as a competitor. Specific competitor DNAs can include the recognition site with one, two, or three nucleotide mutations. Thus, a biochemical assay can be used to measure not only the affinity of a domain for a given site, but also its affinity to the site relative to other sites. Rebar and Pabo (1994) Science 263:671-673 describe a method of obtaining apparent K_{d} constants for zinc finger domains from EMSA.

In one specific example, we inserted the DNA segments encoding zinc finger proteins into pGEX-4T2 (Pharmacia Biotech) between the SalI and NotI sites. Zinc finger proteins were expressed in E. coli strain BL21 as fusion proteins connected to GST (Glutathione-S-transferase). The fusion proteins were purified using glutathione affinity chromatography (Pharmacia Biotech, Piscataway, N.J.) and then digested with thrombin, which cleaves the connecting site between the GST moiety and zinc finger proteins.

Various amounts of a zinc finger protein were incubated with a radioactively labeled probe DNA for one hour at room temperature in 20 mM Tris pH 7.7, 120 mM NaCl, 5 mM MgCl₂, 20 μM ZnSO₄, 10% glycerol, 0.1% Nonidet P-40, 5 mM DTT, and 0.10 mg/mL BSA (bovine serum albumin), and then the reaction mixtures were subjected to gel electrophoresis. The radioactive signals were quantified by PHOSPHORIMAGER™ analysis (Molecular Dynamics), and dissociation constants (K_{d}) were determined as described (Rebar and Pabo (1994) Science 263:671-673).

It is possible to determine the DNA binding affinity of a DNA binding domain whether or not it is associated with (e.g., is covalently attached to) a PTD. For some implementations, it is useful to compare the DNA binding affinity of the domain in the presence of the PTD with the affinity in the absence of the PTD. DNA binding domains that bind with an affinity of less than 50, 10, 5, or 1 nM may be particularly useful. Further DNA binding domains that can discriminate between a target site and a non-target site that is between 70-90% identical are also useful. Such domains can discriminate by a factor of at least 2-, 5-, 10- or 100-fold.

SELEX. SELEX (Systematic Evolution of Ligands by EXponential enrichment) is a method of amplifying nucleic acids that are specifically recognized by a DNA binding domain. First, template oligonucleotides that include a 20-nucleotide long random region flanked by conserved sequences at the ends are prepared. The template oligonucleotides are converted to double-stranded DNA by extension with the Klenow fragment of DNA polymerase and a primer that anneals to the conserved 3′ end. The population of double-stranded DNA is then incubated with the DNA binding domain. For example, 100 μg of protein fused to GST can be mixed with 10 pmol of double-stranded template DNA in 100 μl of binding buffer (25 mM Hepes pH 7.9, 40 mM KCl, 3 mM MgCl₂, 1 mM DTT) for one hour at room temperature. GST-resin (10 μl) is then added to the mixture. After incubation for 30 min at room temperature, the resin is washed three times with binding buffer containing 2.5% skim milk. The bound double-stranded template oligomers are dissociated by incubating the resins with 100 μl of 1 M KCl for 10 min at room temperature. The eluted material is amplified by PCR. The amplified nucleic acid can be used for additional rounds of SELEX. For example, eight rounds of SELEX can be performed prior to cloning and sequencing. Application of SELEX to zinc finger proteins is described, e.g., in U.S. Ser. No. 60/431,892, filed Dec. 9, 2002.

Cellular assays. The function of a transducible DNA binding protein can then be assayed in vivo using a reporter gene. The reporter gene is engineered to include a DNA target site that the DNA binding protein specifically recognizes at a regulatory position, e.g., a position comparable to the position of the Zif268 site in the construct of Kim and Pabo (1997) J Biol Chein 272:29795-29800). After contacting the transducible DNA binding protein with the cell, luciferase reporter activity is evaluated. See also Example 4. It is also possible to express the transducible DNA binding protein within cells in order to specifically assay the DNA binding and transcriptional regulatory functions of the protein and then to use other assays to evaluate whether the protein can be transduced into the cell.

The ability of a transducible DNA binding protein to regulate endogenous genes can also be evaluated, by assaying for transcription of the endogenous gene after contacting the cell with the transducible DNA binding protein or after expressing the transducible DNA binding protein in the cell. Methods of assaying transcription of endogenous genes include Northern analysis, RT-PCR and transcriptional profiling (described below).

Still another method for evaluating the ability of a transducible DNA binding protein to regulate endogenous genes is contact the transducible DNA binding protein with cells and evaluate a parameter of the cell, e.g., a parameter known to be affected by an endogenous gene. For example, it is possible to contact a transducible DNA binding protein that binds to the VEGF promoter to cells and then evaluate the cell for ability to produce VEGF.

Stability. A variety of methods are available to determine the stability of a transducible DNA binding protein in a cell. For example, the protein may be labeled (e.g., with a radioisotope) and then contacted to the cell. The amount of label present in the cell can be monitored as a function of time. Cells can be washed prior to each time point to remove label that is released by protein degradation. Alternatively, samples of contacted cells can be prepared at each time point and electrophoresed on a gel to accurately detect full length protein. In another method, the protein is not labeled by is detected at different time points, e.g., using an antibody that specifically recognizes a protein. The protein may include an epitope tag.

To produce a stable protein it is also useful to inspect the amino acid sequence for degradation signals (e.g., the “PEST” signal) or ubiquitination sites. Those signals and sites can be modified in order to increase stability of the protein. In some special embodiments, it may be desirable to have an unstable protein in which case such signals and sites can be retained or deliberately introduced.

Profiling Reglatory Properties of a Chimeric Zinc Finger Protein

A chimeric zinc finger protein can be characterized to determine its ability to regulate an endogenous gene of a cell, e.g., a mammalian cell. Nucleic acid encoding the chimeric zinc finger protein is first fused to a repression or activation domain, and then introduced into a cell of interest. After appropriate incubation and induction of expression of the coding nucleic acid, mRNA is harvested from the cell and analyzed using a nucleic acid microarray.

Nucleic acid microarrays can be fabricated by a variety of methods, e.g., photolithographic methods (see, e.g., U.S. Pat. No. 5,510,270), mechanical methods (e.g., directed-flow methods as described in U.S. Pat. No. 5,384,261), and pin based methods (e.g., as described in U.S. Pat. No. 5,288,514). The array is synthesized with a unique capture probe at each address, each capture probe being appropriate to detect a nucleic acid for a particular expressed gene.

The mRNA can be isolated by routine methods, e.g., including DNase treatment to remove genomic DNA and hybridization to an oligo-dT coupled solid substrate (e.g., as described in Current Protocols in Molecular Biology, John Wiley & Sons, N.Y). The substrate is washed, and the mRNA is eluted. The isolated mRNA is then reverse transcribed and optionally amplified, e.g., by RT-PCR, e.g., as described in U.S. Pat. No. 4,683,202. The nucleic acid can be labeled during amplification or reverse transcription, e.g., by the incorporation of a labeled nucleotide. Examples of preferred labels include fluorescent labels, e.g., red-fluorescent dye Cy5 (Amersham) or green-fluorescent dye Cy3 (Amersham). Alternatively, the nucleic acid can be labeled with biotin, and detected after hybridization with labeled streptavidin, e.g., streptavidin-phycoerythrin (Molecular Probes).

The labeled nucleic acid is then contacted with the array. In addition, a control nucleic acid or a reference nucleic acid can be contacted with the same array. The control nucleic acid or reference nucleic acid can be labeled with a label other than the sample nucleic acid, e.g., one with a different emission maximum. Labeled nucleic acids are contacted to an array under hybridization conditions. The array is washed and then imaged to detect fluorescence at each address of the array.

Information from imaging the array can be used to generate a profile. For example, the extent of hybridization at an address is represented by a numerical value and stored, e.g., in a vector, a one-dimensional matrix, or one-dimensional array. The vector x has a value for each address of the array. For example, a numerical value for the extent of hybridization at a particular address is stored in variable xₐ. The numerical value can be adjusted, e.g., for local background levels, sample amount, and other variations. Nucleic acid is also prepared from a reference sample and hybridized to the same or a different array. The vector y is construct identically to vector x. The sample expression profile and the reference profile can be compared, e.g., using a mathematical equation that is a function of the two vectors. The comparison can be evaluated as a scalar value, e.g., a score representing similarity of the two profiles. Either or both vectors can be transformed by a matrix in order to add weighting values to different genes detected by the array.

The expression data can be stored in a database, e.g., a relational database such as a SQL database (e.g., Oracle or Sybase database environments). The database can have multiple tables. For example, raw expression data can be stored in one table, wherein each column corresponds to a gene being assayed, e.g., an address or an array, and each row corresponds to a sample. A separate table can store identifiers and sample information, e.g., the batch number of the array used, date, and other quality control information.

Genes that are similarly regulated can be identified by clustering expression data to identify coregulated genes. Such cluster may be indicative of a set of genes coordinately regulated by the chimeric zinc finger protein. Genes can be clustered using hierarchical clustering (see, e.g., Sokal and Michener (1958) Univ. Kans. Sci. Bull. 38:1409), Bayesian clustering, k-means clustering, and self-organizing maps (see, Tamayo et al. (1999) Proc. Natl. Acad. Sci. USA 96:2907).

The similarity of a sample expression profile to a reference expression profile (e.g., a control cell) can also be determined, e.g., by comparing the log of the expression level of the sample to the log of the predictor or reference expression value and adjusting the comparison by the weighting factor for all genes of predictive value in the profile.

Targets for Gene Regulation

One or more genes in a cell can be the target of gene regulation. For example, a gene required by a pathogen can be repressed, a gene required for cancerous growth can be repressed, a gene poorly expressed or encoding an unstable protein can be activated and overexpressed, and so forth. Examples of specific target genes include genes that encode: cell surface proteins (e.g., glycosylated surface proteins), cancer-associated proteins, tumor suppressors, cytokines, chemokines, peptide hormones, neurotransmitters, cell surface receptors, cell surface receptor kinases, seven transmembrane receptors, virus receptors and co-receptors, extracellular matrix binding proteins, cell-binding proteins, antigens of pathogens (e.g., bacterial antigens, malarial antigens, and so forth). Additional protein targets include enzymes such as enolases, cytochrome P450s, acyltransferases, methylases, TIM barrel enzymes, isomerases, acyl transferases, and so forth.

More specific examples include: integrins, cell attachment molecules or “CAMs” such as cadherins, selectins, N-CAM, E-CAM, U-CAM, I-CAM and so forth); proteases (e.g., subtilisin, trypsin, chymotrypsin; a plasminogen activator, such as urokinase or human tissue-type plasminogen activator); bombesin; factor IX, thrombin; CD-4; platelet-derived growth factor; insulin-like growth factor-I and -II; nerve growth factor; fibroblast growth factor (e.g., aFGF and bFGF); epidermal growth factor (EGF); VEGFa; pigment epithelium-derived factor (PEDF); transforming growth factor (TGF, e.g., TGF-α and TGF-β; insulin-like growth factor binding proteins; brain-derived neurotrophic factor (BDNF); erythropoietin; thrombopoietin; mucins; human serum albumin; lectin; growth hormone (e.g., human growth hormone); proinsulin; insulin A-chain; insulin B-chain; parathyroid hormone; thyroid stimulating hormone; thyroxine; follicle stimulating hormone; calcitonin; atrial natriuretic peptides A, B or C; leutinizing hormone; glucagon; factor VIII; hematopoietic growth factor; tumor necrosis factor (e.g., TNF-α and TNF-β); enkephalinase; jun B proto-oncogene; protein kinase C; brain-specific Na-dependent inorganic phosphate cotransporter; cellular retinoic acid-binding protein 1; cellular retinoic acid-binding protein 2; differentiation-related gene-1 (Drg-1); Transcription factor E2F; Early growth response-1 (EGR-1); protein tyrosine phosphatases 1B (PTP-1B); Fas; melanoma differentiation associated gene-7 (MDA-7); presenilin-1 (PS-1); angiotensin converting enzyme; Angiopoietin-2; b-secretase (BACE1); mmp3; checkpoint with forkhead associated and ring finger (CHFR); peroxisome proliferator-activated receptor gamma (PPAR-gamma); TNF-related apoptosis-inducing ligand (TRAIL); Ku-80; ataxia-telangiectasia mutated (ATM); BRCA; CC-chemokine receptor 5 (CCR5); tumor necrosis factor alpha-induced protein-3 (TNFAIP3); c-myc, Hypoxia-inducible factor −1 alpha (HIF-1 alpha); caspase-3; intercellular adhesion molecule type I (ICAM-1); angiotensin II receptor 1 (AT-1R); Mullerian-inhibiting substance; gonadotropin-associated peptide; tissue factor protein; inhibin; activin; receptors for hormones or growth factors; rheumatoid factors; osteoinductive factors; an interferon, e.g., interferon-α, β, γ; colony stimulating factors (CSFs), e.g., M-CSF, GM-CSF, and G-CSF; interleukins (ILs), e.g., IL-1, IL-2, IL-3, IL-4, IL-12, and IL-13 etc.; decay accelerating factor; and immunoglobulins. In some embodiments, the target protein is associated with a disease, e.g., cancer, an infectious disease, or a cardiovascular disease.

Some target genes may be encoded by a foreign genome or other nucleic acid that is introduced into a cell, e.g., the genome of a retrovirus, a gene therapy vector, a DNA virus and so forth.

Producing a Transducible Transcription Factor

Standard recombinant nucleic acid methods can be used to express a transducible DNA binding protein. In one embodiment, a nucleic acid sequence encoding the transducible protein is cloned into a nucleic acid expression vector, e.g., with appropriate signal and processing sequences and regulatory sequences for transcription and translation. In another embodiment, the protein can be synthesized using automated organic synthetic methods. Synthetic methods for producing proteins are described, for example in Methods in Enzymology, Volume 289: Solid-Phase Peptide Synthesis by Gregg B. Fields (Editor), Sidney P. Colowick, Melvin I. Simon (Editor), Academic Press; (Nov. 15, 1997) ISBN: 0121821900.

The expression vector for expressing the transducible protein can include regulatory sequences, including for example, a promoter, operably linked to sequence encoding the transducible protein. Non-limiting examples of inducible promoters that can be used include steroid-hormone responsive promoters (e.g., ecdysone-responsive, estrogen-responsive, and glutacorticoid-responsive promoters), the tetracyclin “Tet-On” and “Tet-Off” systems, and metal-responsive promoters. The construct can be introduced into an appropriate host cell, e.g., a bacterial cell, yeast cell, insect cell, or tissue culture cell. The construct can also be introduced into embryonic stem cells to generate a transgenic organism as a model subject. Large numbers of suitable vectors and promoters are known to those of skill in the art and are commercially available for generating the recombinant constructs of the present invention.

Known methods can be used to construct vectors containing a polynucleotide of the invention and appropriate transcriptional/translational control signals. These methods include in vitro recombinant DNA techniques, synthetic techniques and in vivo recombination/genetic recombination. See, for example, the techniques described in Sambrook & Russell, Molecular Cloning: A Laboratory Manual, 3^{rd} Edition, Cold Spring Harbor Laboratory, N.Y. (2001) and Ausubel et al., Current Protocols in Molecular Biology (Greene Publishing Associates and Wiley Interscience, N.Y. (1989).

Host cells suitable for producing a transducible protein include bacterial cells and eukaryotic cells (e.g., fungal, insect, plant, and mammalian cells). Host cells can be disrupted by any convenient method, including freeze-thaw cycling, sonication, mechanical disruption, or use of cell lysing agents. Scopes (1994) Protein Purification: Principles and Practice, New York:Springer-Verlag provides a number of general methods for purifying recombinant (and non-recombinant) proteins. The method can include, e.g., ion-exchange chromatography, size-exclusion chromatography, affinity chromatography, selective precipitation, dialysis, and hydrophobic interaction chromatography. These methods can be adapted for devising a purification strategy for the transducible protein. If the transducible protein includes a purification handle such as an epitope tag or a metal chelating sequence, affinity chromatography can be used to highly purify the protein.

The amount of protein produced can be evaluated by detecting the transducible DNA binding protein directly (e.g., using Western analysis) or indirectly (e.g., by assaying materials from the cells for specific DNA binding activity, e.g., by EMSA). Protein can be detected prior to purification, during any stage of purification, or after purification. In some implementations, purification or complete purification may not be necessary.

In addition to use in protein transduction, a transducible DNA binding protein can be produced in a subject cell or subject organism in order to regulate an endogenous gene. The transducible DNA binding protein can be configured, as described above, to bind to a region of the endogenous gene and to provide a transcriptional activation or repression function. As described in Kang and Kim (supra), the expression of a nucleic acid encoding the designed protein can be operably linked to an inducible promoter. By modulating the concentration of the inducer for the promoter, the expression of the endogenous gene can be regulated in a concentration dependent manner.

In another example, the transducible DNA binding protein is produced as a secreted protein by one cell so that the protein can diffuse and enter another cell in which it causes an alteration in gene regulation. The diffusion may occur within a subject, e.g., from one cell of a subject to another.

Cell Targeting Moieties

A transducible DNA binding protein can include a cell targeting moiety that is specific for one or more specific cell types (e.g., one or more particular differentiated cells or affected cells), or one or more cell states (e.g., a proliferative state). For example, the cell targeting moiety can be specific for disease state, a differentiated state, or a proliferative state. Example of cell targeting moieties include proteins such as antibodies or cell receptor recognition peptides.

Some antigens that can be specifically recognized by a cell targeting moieties include tumor or cancer cell specific antigens. Exemplary antigens include: tumor-associated glycoprotein (TAG72), Carcinoembryonic antigen (CEA), 180 kDa glycoprotein polymorphic epithelial mucin HMFG1 (PEM or MUC1), Epithelial membrane antigen (EMA), epidermal growth factor receptor (EGFR), HER2/c-erb-B2, Prostate-specific membrane antigen (PSMA), CD33, and CD20. Antibody fragments can be prepared by phage-display technology or by immunization. Various methods of producing, modifying, assaying and using antibodies are described in Antibodies: A Laboratory Manual, Harlow and Lane (eds.), Cold Spring Harbor Laboratory Press, 1988; Alting-Mees et al., “Monoclonal Antibody Expression Libraries: A Rapid Alternative to Hybridomas”, Strategies in Molecular Biology 3:1-9 (1990); and Larrick et al., Biotechnology, 7:394 (1989).

In one embodiment, the cell targeting moiety includes a tumor homing peptide. Examples of tumor homing peptides are described in PCT/US00/01602 and US Published Application No. 2001-0046498.

In another embodiment, the cell targeting moiety includes a naturally occurring polypeptide which interacts with a cell-specific protein. For example, the naturally occurring polypeptide can include all or part of a domain of a growth hormone, cytokine, or other protein that specifically interacts with a cell surface protein, e.g., a cell surface receptor.

Pharmaceutical Compositions

In another aspect, the invention provides compositions, e.g., pharmaceutically acceptable compositions, which include a transducible, artificial DNA binding protein, e.g., a transducible, artificial zinc finger protein or another protein described herein, formulated together with a pharmaceutically acceptable carrier. As used herein, “pharmaceutical compositions” encompass diagnostic as well as therapeutic compositions.

As used herein, “pharmaceutically acceptable carrier” includes any and all solvents, dispersion media, coatings, antibacterial and antifungal agents, isotonic and absorption delaying agents, and the like that are physiologically compatible. Generally, the carrier is suitable for intravenous, intramuscular, subcutaneous, parenteral, spinal or epidermal administration (e.g., by injection or infusion). Depending on the route of administration, the transducible DNA binding protein may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

In one embodiment, a transducible, artificial DNA binding protein can be formulated for sustained release. For example, the transducible DNA binding protein can be encapsulated in a matrix, e.g., a lipid-protein-sugar matrix for delivery to an individual. The encapsulated transducible DNA binding protein can be formed into small particles, in a size ranging from 5 micrometers to 50 nanometers. The lipid-protein-sugar particles typically include a surfactant or phospholipid or similar hydrophobic or amphiphilic molecule; a protein; a simple and/or complex sugar; and the transducible, artificial DNA binding protein. In one example, the lipid is dipalmitoylphosphatidylcholine (DPPC), the protein is albumin, and the sugar is lactose. In another example, a synthetic polymer is substituted for at least one of the components of the lipid-protein-sugar particle, e.g., the lipid, protein, and/or sugar. The compounds used to create LPSPs can be naturally occurring and therefore have improved biocompatibility. The particles may be prepared using techniques known in the art including spray drying. See, e.g., U.S Published application 2002-0150621

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form can depend on the intended mode of administration and therapeutic application. The composition may include an anionic or negatively charged carrier, e.g., to stabilize the composition. For example, the composition may include small fragments of poly-A-polyT DNA duplexes.

Typical compositions are in the form of injectable or infusible solutions, such as compositions similar to those used to administer antibodies to human subjects. The typical mode of administration is parenteral (e.g., intravenous, subcutaneous, intraperitoneal, intramuscular). In one embodiment, the transducible, artificial DNA binding protein is administered by intravenous infusion or injection. In another embodiment, the transducible, artificial DNA binding protein is administered by intramuscular or subcutaneous injection. Additional exemplary routes of administration include oral administration, application to epidermal tissue (e.g., skin) or a mucosa (e.g., the eye) and inhalation. Other routes of administration are also possible.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions typically must be sterile and stable under the conditions of manufacture and storage. A pharmaceutical composition can also be tested to insure it meets regulatory and industry standards for administration. For example, endotoxin levels in the preparation can be tested using the Limulus amebocyte lysate assay (e.g., using the kit from Bio hittaker lot # 7L3790, sensitivity 0.125 EU/mL) according to the USP 24/NF 19 methods. Sterility of pharmaceutical compositions can be determined using thioglycollate medium according to the USP 24/NF 19 methods. For example, the preparation is used to inoculate the thioglycollate medium and incubated at 35° C. for 14 or more days. The medium is inspected periodically to detect growth of a microorganism.

The composition can be formulated as a solution, microemulsion, dispersion, liposome, or other ordered structure suitable to high drug concentration. Sterile injectable solutions can be prepared by incorporating the active compound (i.e., the transducible DNA binding protein) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, typical methods of preparation include vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The transducible, artificial DNA binding proteins can be administered by a variety of methods known in the art, although for many applications, the route/mode of administration is intravenous injection or infusion. For example, for therapeutic applications, the transducible, artificial DNA binding protein can be administered by intravenous infusion at a rate of less than 30, 20, 10, 5, 3, 1, or 0.1 mg/min to reach a dose of about 1 to 100 mg/m², 7 to 25 mg/m², or 0.5 to 15 mg/m². The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

In certain embodiments, the transducible DNA binding protein may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by a route other than parenteral administration, it may be useful to coat the compound with, or co-administer the compound with, a material to prevent its inactivation.

Pharmaceutical compositions can be administered with medical devices known in the art. For example, a pharmaceutical composition can be administered with a needleless hypodermic injection device, such as the devices disclosed in U.S. Pat. No. 5,399,163. Examples of well-known implants and modules that can be used include: U.S. Pat. No. 4,487,603, which discloses an implantable micro-infusion pump for dispensing medication at a controlled rate; U.S. Pat. No. 4,486,194, which discloses a therapeutic device for administering medications through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are also known.

In one embodiment, the transducible DNA binding protein is administered intravenously and enters a neuronal cell, e.g., a brain cell. For example, the transducible DNA binding protein is physically associated with a protein that binds to a neuronal cell, e.g., a brain cell, e.g., glial line-derived neurotrophic factor (GNDF). In another embodiment, the transducible DNA binding protein is contacted to a hematopoietic cell, e.g., a lymphocyte, e.g., a T cell, and regulates gene expression in that cell. For example, the protein can be used to regulate gene expression in specific cytotoxic T cells, e.g., for treatment of infectious disease and cancer.

In certain embodiments, a transducible, artificial DNA binding protein can be formulated to ensure proper distribution in vivo. For example, the blood-brain barrier (BBB) excludes many highly hydrophilic compounds. To ensure that a transducible, artificial DNA binding protein can cross the BBB (if desired), it can be formulated, for example, in liposomes. For methods of manufacturing liposomes, see, e.g., U.S. Pat. Nos. 4,522,811; 5,374,548; and 5,399,331. The liposomes may include one or more moieties which are selectively transported into specific cells or organs, thus enhance targeted drug delivery (see, e.g., V. V. Ranade (1989) J. Clin. Pharmacol. 29:685).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit can be dictated by and directly dependent on (a) the unique characteristics of the active compound and the particular therapeutic effect to be achieved, and (b) the limitations inherent in the art of compounding such an active compound for the treatment of sensitivity in individuals.

Dosage values may vary with the type and severity of the condition to be alleviated or prevented. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Pharmaceutical compositions may include a “therapeutically effective amount” or a “prophylactically effective amount” of a transducible, artificial DNA binding protein. A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the transducible DNA binding protein to elicit a desired response in the individual. A therapeutically effective amount is also one that provides a therapeutically beneficial effect, and typically one in which any toxic or detrimental effects of the composition is outweighed by the beneficial effect. For example, a transducible, artificial DNA binding protein can inhibit a measurable parameter, e.g., the rate of growth of a cancer cell. A measurable parameter can be evaluated in any subject, e.g., an animal model system predictive of efficacy in humans or in a human subject, e.g., a patient, control subject and so forth. Alternatively, this property of a composition can be evaluated by examining the ability of the compound to produce a desired effect, e.g., inhibit cell growth or change (e.g., increase or decrease) levels of a protein, such as a cytokine or growth factor. Many assays for such measurable parameters are known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the invention are kits that include the transducible, artificial DNA binding protein and instructions for use, e.g., treatment, prophylactic, or diagnostic use. In one embodiment, the instructions for therapeutic applications include suggested dosages and/or modes of administration in a patient with a disease, disorder, or condition. The kit can further contain a least one additional reagent, such as a diagnostic or therapeutic agent, e.g., a diagnostic or therapeutic agent as described herein, and/or one or more additional transducible agents, formulated as appropriate, in one or more separate pharmaceutical preparations.

In one embodiment, a transducible, artificial DNA binding protein is physically associated with a moiety that improves its stabilization and/or retention in circulation, e.g., in blood, serum, lymph, or other tissues. The moiety can improve circulatory half-life by at least two, four, or six fold. For example, a transducible, artificial DNA binding protein can be associated with a polymer, e.g., a substantially non-antigenic polymers, such as polyalkylene oxides or polyethylene oxides. Suitable polymers will vary substantially by weight. Polymers having molecular number average weights ranging from about 200 to about 35,000 are usually selected. Molecular weights of from about 1,000 to about 15,000 or from about 2,000 to about 12,500 can be used. In one embodiment, the polymer is attached by a reversible bond that is broken when the transducible DNA binding protein enters a cell. For example, a disulfide or other thiol bond can be reduced in the cell to separate the polymer from the transducible DNA binding protein.

For example, a transducible, artificial DNA binding protein can be conjugated to a water soluble polymer, e.g., hydrophilic polyvinyl polymers, e.g. polyvinylalcohol and polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides, heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon. Other compounds can also be attached to the same polymer, e.g., a label or a targeting agent.

In one embodiment, the polymer is frequently water soluble prior to cross-linking. Generally, after crosslinking, the product is water soluble, e.g., exhibits a water solubility of at least about 0.01 mg/ml, 0.1 mg/ml, or 1 mg/ml. In addition, the polymer should not be highly immunogenic in the conjugate form, nor should it possess viscosity that is incompatible with intravenous infusion or injection if the conjugate is intended to be administered by such routes. The molecular weight of the polymer can range up to about 500,000 D, e.g., at least about 20,000 D, 30,000 D, or 40,000 D.

The covalent crosslink can be used to attach a transducible, artificial DNA binding protein to a polymer, for example, crosslinking to the N-terminal amino group and epsilon amino groups found on lysine residues, as well as other amino, imino, carboxyl, sulfhydryl, hydroxyl or other hydrophilic groups. Functionalized PEG polymers that can be attached to a transducible, artificial DNA binding protein are available, e.g., from Shearwater Polymers, Inc. (Huntsville, Ala.).

The conjugates of a transducible, artificial DNA binding protein and a polymer can be separated from the unreacted starting materials, e.g., by gel filtration or ion exchange chromatography, e.g., HPLC. Heterologous species of the conjugates are purified from one another in the same fashion. Resolution of different species (e.g. containing one or two PEG residues) is also possible due to the difference in the ionic properties of the unreacted amino acids. See, e.g., WO 96/34015.

Storage. A variety of methods can be used to store purified transducible zinc finger proteins. For example, the proteins can be stored in the presence of one or more of (i) a cryoprotectant (e.g., glycerol, e.g., between 5-12% glycerol), (ii) zinc, e.g., 1 μM to 5 mM, 1 μM to 500 μM, 1 μM to 200 μM, 0.05 μM to 50 μM, and 0.5 μM to 30 μM zinc, and (iii) a reducing agent (e.g., DTT, e.g., about 0.05-5 mM, e.g., 0.5-2 mM). The proteins can be stored e.g., at 4° C. or less, e.g., −20° C. or less, e.g., between about 60° C. to −90° C.

Treatments

Transducible DNA binding proteins that can regulate an endogenous gene, particularly proteins that can regulate the VEGF-A gene, have therapeutic and prophylactic utilities. For example, a transducible zinc finger protein can be administered to cells in culture, e.g. in vitro or ex vivo, or in a subject, e.g., in vivo, to treat, prevent, and/or diagnose a variety of disorders, such as cancers, particularly metastatic cancers, an inflammatory disorder, and other disorders associated with increased angiogenesis.

As used herein, the term “treat” or “treatment” is defined as the application or administration of a zinc finger protein such that the protein enters cells and regulates gene expression in the cells of a subject, e.g., a patient, or application or administration of the agent to an isolated tissue or cell, e.g., cell line, from a subject, e.g., a patient, who has a disorder (e.g., a disorder as described herein), a symptom of a disorder or a predisposition toward a disorder, with the purpose to cure, heal, alleviate, relieve, alter, remedy, ameliorate, improve or affect the disorder, the symptoms of the disorder or the predisposition toward the disorder.

In one embodiment, “treating a celr” or “treating a tissue” refers to a reduction in at least one activity of a cell, e.g., VEGF-A production, angiogenesis stimulation, proliferation, or other activity of a cell, e.g., a hyperproliferative cell or cell in or near a tissue, e.g., a tumor. Such reduction can include a reduction, e.g., a statistically significant reduction, in the activity of a cell or tissue (e.g., metastatic tissue) or the number of the cell or size of the tissue, the amount or degree of blood supply to the tissue. An example of a reduction in activity is a reduction in migration of the cell (e.g., migration through an extracellular matrix), a reduction in blood vessel formation, or a reduction in cell differentiation. Another example is an activity that, directly or indirectly, reduces inflammation or an indicator of inflammation.

As used herein, an amount of a transducible zinc finger protein effective to treat a disorder, or a “therapeutically effective amount” refers to an amount of the protein which is effective, upon single or multiple dose administration to a subject, in treating a cell.

As used herein, an amount of a transducible zinc finger protein effective to prevent a disorder, or a “a prophylactically effective amount” of the protein refers to an amount of the protein, which is effective, upon single- or multiple-dose administration to the subject, in preventing or delaying the occurrence of the onset or recurrence of a disorder, e.g., a cancer, angiogenesis-based disorder, or inflammatory disorder.

As used herein, the term “subject” is intended to include human and non-human animals. Exemplary subjects include a human patient having a disorder characterized by abnormal cell proliferation or cell differentiation. The term “non-human animals” includes all non-human vertebrates, e.g., non-mammals (such as chickens, amphibians, reptiles) and mammals, such as non-human primates, sheep, dog, cow, pig, etc.

In one embodiment, the subject is a human subject. In one embodiment, the composition of a transducible zinc finger protein can be administered to a non-human mammal (e.g., a primate, pig or mouse) for veterinary purposes or as an animal model of human disease. Regarding the latter, such animal models may be useful for evaluating the therapeutic efficacy of the composition (e.g., testing of dosages and time courses of administration).

In one embodiment, the invention provides a method of treating a neoplastic disorder. The method can include the steps of contacting a cell of a subject with a transducible zinc finger protein, e.g., a zinc finger protein that regulates VEGF-A, in an amount sufficient to treat or prevent the neoplastic disorder. The protein includes a protein transduction domain. For example, the disorder can be caused by a cancerous cell, a tumor cell or a metastatic cell. The subject method can be used on cells in culture, e.g. in vitro or ex vivo. For example, cancerous or metastatic cells (e.g., renal, urothelial, colon, rectal, lung, breast, endometrial, ovarian, prostatic, or liver cancerous or metastatic cells) can be cultured in vitro in culture medium and the contacting step can be effected by adding the zinc finger protein to the culture medium. The method can be performed on cells (e.g., cancerous or metastatic cells) present in a subject (e.g., a human subject), as part of an in vivo (e.g., therapeutic or prophylactic) protocol. For in vivo embodiments, the contacting step is effected in a subject and includes administering the transducible zinc finger protein to the subject under conditions effective to permit regulation of the VEGF-A gene in cells of the subject.

The method can be used to treat a cancer. As used herein, the terms “cancer”, “hyperproliferative”, “malignant”, and “neoplastic” are used interchangeably, and refer to those cells an abnormal state or condition characterized by rapid proliferation or neoplasm. The terms include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be benign, premalignant or malignant.

Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx, prostate, ovary as well as adenocarcinomas which include malignancies such as most colon cancers, rectal cancer, renal-cell carcinoma, liver cancer, non-small cell carcinoma of the lung, cancer of the small intestine and so forth. Metastatic lesions of the aforementioned cancers also can be treated or prevented using a method or composition described herein.

The subject method can be useful in treating malignancies of the various organ systems, such as those affecting lung, breast, lymphoid, gastrointestinal (e.g., colon), and genitourinary tract, prostate, ovary, pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is recognized by those skilled in the art and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include choriocarcinomas and those forming from tissue of the cervix, lung, prostate, breast, endometrium, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is recognized by those skilled in the art and refers to malignant tumors of mesenchymal derivation.

The method also can be used to modulate (e.g., increase or inhibit the proliferation of cells of hematopoietic origin shown to express VEGF-A. For example, the method can be used to inhibit the proliferation of hyperplastic/neoplastic cells.

Methods of administering transducible zinc finger proteins are described in “Pharmaceutical Compositions”. Suitable dosages of the molecules used will depend on the age and weight of the subject and the particular drug used.

A transducible zinc finger protein can be coupled to label, e.g., for imaging in a subject after it is delivered to a subject. Suitable labels include MRI-detectable labels or radiolabels.

A transducible zinc finger protein can be administered alone or in combination with one or more of the existing modalities for treating cancers, including, but not limited to: surgery; radiation therapy, and chemotherapy. For example, the transducible zinc finger protein can be administered with another anti-angiogenic agent. Exemplary anti-angiogenic agents include: 2ME2, Angiostatin, Angiozyme, Anti-VEGF RhuMAb, Apra (CT-2584), Avicine, Benefin, BMS275291, Carboxyamidotriazole, CC4047, CC5013, CC7085, CDC801, CGP-41251 (PKC 412), CM101, Combretastatin A-4 Prodrug, EMD 121974, Endostatin, Flavopiridol, Genistein (GCP), Green Tea Extract, IM-862, ImmTher, Interferon alpha, Interleukin-12, Iressa (ZD 1839), Marimastat, Metastat (Col-3), Neovastat, Octreotide, Paclitaxel, Penicillamine, Photofrin, Photopoint, PI-88, Prinomastat (AG-3340), PTK787 (ZK22584), R0317453, Solimastat, Squalamine, SU 101, SU 5416, SU-6668, Suradista (FCE 26644), Suramin (Metaret), Tetrathiomolybdate, Thalidomide, TNP-470 and Vitaxin additional antiangiogenic agents are described by Kerbel, J. Clin. Oncol. 19(18s):45s-51s, 2001.

To treat a neoplastic disorder, the protein can be administered in combination with one or more the following: Examples of other therapeutic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol or DM1 (see U.S. Pat. No. 5,208,020), CC-1065 (see U.S. Pat. Nos. 5,475,092, 5,585,499, 5,846,545) calicheamicin, and analogs or homologs thereof. The term “in combination” in this context means that different agents are given substantially contemporaneously, either simultaneously or sequentially. If given sequentially, at the onset of administration of the second compound, the first of the two agents is preferably still detectable at effective concentrations at the site of treatment.

A transducible zinc finger protein, particularly one that can regulate (e.g., reducing expression of) the VEGF-A gene, can be administered alone or in combination with one or more of the existing modalities for treating an inflammatory disease or disorder. Exemplary inflammatory diseases or disorders include: acute and chronic immune and autoimmune pathologies, such as, but not limited to, rheumatoid arthritis (RA), juvenile chronic arthritis (JCA), psoriasis, graft versus host disease (GVHD), scleroderma, diabetes mellitus, allergy; asthma, acute or chronic immune disease associated with an allogenic transplantation, such as, but not limited to, renal transplantation, cardiac transplantation, bone marrow transplantation, liver transplantation, pancreatic transplantation, small intestine transplantation, lung transplantation and skin transplantation; chronic inflammatory pathologies such as, but not limited to, sarcoidosis, chronic inflammatory bowel disease, ulcerative colitis, and Crohn's pathology or disease; vascular inflammatory pathologies, such as, but not limited to, disseminated intravascular coagulation, atherosclerosis, Kawasali's pathology and vasculitis syndromes, such as, but not limited to, polyarteritis nodosa, Wegener's granulomatosis, Henoch-Schonlein purpura, giant cell arthritis and microscopic vasculitis of the kidneys; chronic active hepatitis; Sjogren's syndrome; psoriatic arthritis; enteropathic arthritis; reactive arthritis and arthritis associated with inflammatory bowel disease; and uveitis.

Inflammatory bowel diseases (IBD) include generally chronic, relapsing intestinal inflammation. IBD refers to two distinct disorders, Crohn's disease and ulcerative colitis (UC). The clinical symptoms of IBD include intermittent rectal bleeding, crampy abdominal pain, weight loss and diarrhea. A clinical index can also be used to monitor IBD such as the Clinical Activity Index for Ulcerative Colitis. See also, e.g., Walmsley et al. Gut. 1998 July;43(1):29-32 and Jowett et al. (2003) Scand J Gastroenterol. 38(2):164-71.

A transducible zinc finger protein can be used to treat or prevent one of the foregoing diseases or disorders. For example, the protein can be administered (locally or systemically) in an amount effective to ameliorate at least one symptom of the respective disease or disorder. The protein may also ameliorate inflammation, e.g., an indicator of inflammation, e.g., such as local temperature, swelling (e.g., as measured), redness, local or systemic white blood cell count, presence or absence of neutrophils, cytokine levels, and so forth. It is possible to evaluate a subject, e.g., prior, during, or after administration of the protein, for one or more of indicators of inflammation, e.g., an aforementioned indicator.

A transducible zinc finger protein, particularly one that can regulate (e.g., increase expression of) the VEGF-A gene, can be administered alone or in combination with one or more of the existing modalities for treating a wound, e.g., to promote wound healing. For example, generally, activation of VEGF-A can increase formation of new blood vessels and capillaries. The protein can also be used for ameliorating surgery, burn, traumas, ulcers, bone fractures, and other disorders that require increased angiogenesis.

A transducible zinc finger protein, particularly one that can regulate (e.g., increase expression of) the VEGF-A gene, can be administered alone or in combination with one or more of the existing modalities for treating a cardiovascular disorder, e.g., e.g., ischemic heart disease, peripheral artery disease, or coronary artery disease. A method of administering zinc finger proteins can also be used to treat diabetic retinopathy or a patient suffering from a myocardial infarct.

Some aspects of transducible DNA binding proteins are further illustrated by the following specific and non-limiting examples.

### EXAMPLE 1

### Construction, Expression and Purification of TAT-ZFP Fusion Proteins

A nucleic acid encoding a chimeric three or four-fingered protein was prepared as follows. The vector P3 (Toolgen, Inc.) was used to express chimeric zinc finger proteins in mammalian cells. P3 was constructed by modification of the pcDNA3 vector (Invitrogen, San Diego Calif.). A synthetic oligonucleotide duplex having compatible overhangs was ligated into the pcDNA3 vector digested with HindIII and XhoI. The duplex contains nucleic acid that encodes the hemagglutinin (HA) tag and a nuclear localization signal. The duplex also includes BamHI, EcoRI and NotI and BglII restriction site sites and a stop codon. Further, the XnaI site in SV40 origin of the resulting vector was destroyed by digestion with XmaI, filling in the overhanging ends of the digested restriction site, and religation of the ends.

The following is one exemplary method for constructing a plasmid that encodes a chimeric zinc finger protein with multiple zinc finger domains as listed in Table 2 above. First, an insert that encodes a single zinc finger domain was inserted into a vector (the P3 vector) that harbored a sequence encoding a single zinc finger domain. The result of this cloning is a plasmid that encodes a zinc finger protein with two zinc finger domains. A zinc finger domain insert consisting of two zinc finger domains was prepared by the above method and cloned into AgeI/NotI-linearized vector P3 having one or two zinc finger domains to obtain a plasmid containing a chimeric zinc finger protein gene consisting of three or four zinc finger domains.

Nucleic acids encoding pre-assembled ZFPs were inserted into pTAT plasmid (see, e.g., Dowdy et al. (1999) Science 285: 1569-1572) as follows. First, KpnI restriction sites were added to the ZFP-coding sequences by PCR using a forward primer containing KpnI site at the 5′ sequence. The inserts and vector (PTAT) were prepared by digestion with KpnI and XhoI. After ligation, pTAT-ZFPs constructs were sequenced. After insertion into the pTAT-ZFP plasmid, a sequence is produced that encodes a polypeptide that includes (from N to C terminus): ATG (start codon), a hexa-histidine tag, the HIV Tat PTD sequence, a nuclear localization signal (NLS) and an array of zinc finger domains with functional domain (p65, KRAB or KOX).

E. coli BL21(DE3) cells were transformed with the pTAT-ZFP plasmid and grown in selective medium until they reach to OD 0.3-0.4. Then the cells were induced with 1 mM IPTG for three hours. The TAT-ZFP protein samples were prepared using Ni-NTA agarose (Qiagen) following manufacturer's general instruction. Briefly, the BL21 cells were spun down and the cell pellets were lysed with lysis buffer (100 mM NaH₂PO₄, 10 mM Tris-Cl, 8 M Urea, pH 8.0), sonicated using five cycles of 10 seconds in which sonication for 10 seconds was followed by a 30 second pause (Fischer Scientific 550). The non-purified lysates were stored for series of experiments or the lysates were purified using an affinity column. The lysates were incubated with Ni-NTA agarose (Qiagen) for 40 minutes and washed twice in wash solution (100 mM NaH₂PO₄, 10 mM Tris-Cl, 8 M urea, pH 6.3). The protein was then eluted in elution buffer (100 mM NaH₂PO₄, 10 mM Tris-Cl, 8 M urea, pH 4.5) and dialyzed in PBS. The purified protein was then stored with 10% glycerol at −70° C.

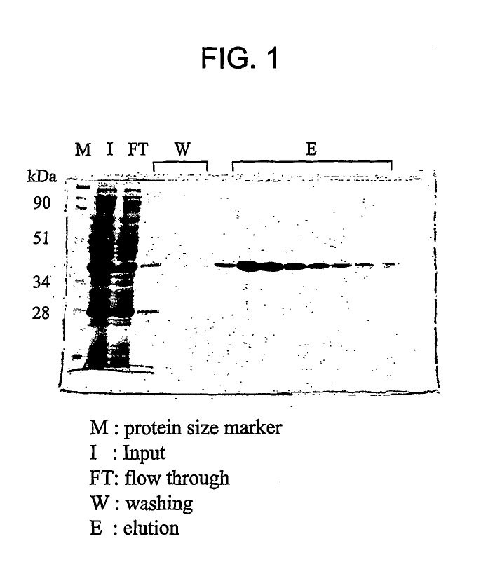

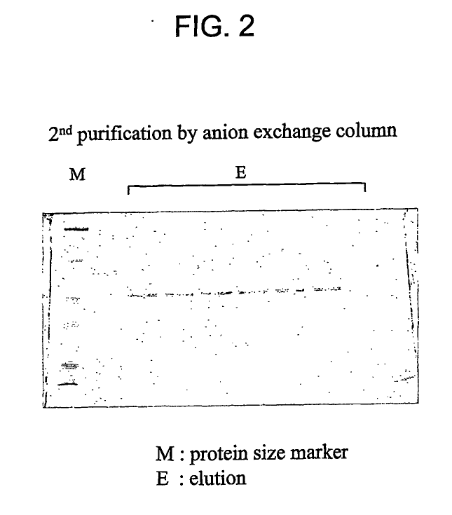

The flow-through obtained at each step in purification and the purified TAT-ZFPs were analyzed on SDS-PAGE gel with Commassie blue staining. The eluted protein migrated as a predominant species in a 35 kDa band and was highly purifie. See FIGS. 1 and 2.

### EXAMPLE 2

### Transduction of TAT-ZFP fusion proteins into mammalian cell culture (1) Evaluation of Transduction Efficiency of TAT Fusion Protein

To determine efficiency of delivery of TAT fusion protein into cultured cells, we used purified TAT-lacZ protein. Genetic TAT-lacZ fusions were generated by insertion of the lacZ open reading frame DNA into pTAT-HA plasmids. See, e.g., Dowdy et al. (1999) Science 285:1569-1572, and they were then transformed into BL21(DE3)LysS bacteria (Novagen). Expression of TAT-lacZ fusion protein was induced by 1 mM IPTG for three hours, the BL21 (DE3)LysS cells were spun down and the cell pellets were lysed with lysis buffer (100 mM NaH₂PO₄, 10 mM Tris-Cl, 8 M Urea, pH 8.0), sonicated using five cycles of 10 seconds in which sonication for 10 seconds was followed by a 30 second pause (Fischer Scientific 550). The lysates were incubated with Ni-NTA agarose (Qiagen) for 40 minutes and washed twice in wash solution (100 mMNaH₂PO₄, 10 mM Tris-Ci, 8 M urea, pH 6.3). The protein was then eluted in elution buffer (100 mM NaH₂PO₄, 10 mM Tris-Ci, 8 M urea, pH 4.5) and dialyzed in PBS. The purified protein was then stored with 10% glycerol at −70° C. Human embryonic kidney (HEK) 293 cells were seeded at 3×10⁵/ml in 24-well culture plates and incubated for 24 hours prior to contact with the TAT fusion proteins. Then, the total lysate obtained from an E. coli culture expressing TAT-lacZ was added to the culture media at concentrations ranging from 100 μg/ml to 800 μg/ml and incubated for 4 hours at 37° C. The cells were washed twice with PBS and fixed with 2% paraformaldehyde, then stained with X-gal. The observed staining intensity was dose dependent. We observed transduction efficiencies of ˜100% of the cells at even the lowest concentration (100 μg/ml). These results indicate that the TAT-domain can transduce high molecular weight proteins (˜120 kD) such as lacZ into cells.

(2) Transduction of TAT-ZFP Fusion Proteins into Cultured Cells

The purified TAT-ZFP fusion proteins were tested for transduction into HEK 293 cells. The HEK 293 cells were pre-cultured for 24 hours at density of 3×10⁵/ml on 24-well culture plates, then 20 μg of TAT-ZFPs were added to culture media. The cells were harvested, washed three times with PBS, then added with ice-cold lysis buffer. The samples were run on SDS-PAGE and the gel was transferred onto a HYBOND-P™ membrane (Amersham Pharmacia Biotech). Western analysis was performed using mouse anti-HA antibody as primary antibody, anti-mouse IgG-HRP as secondary antibody and revealed by ECL™ (Amersham Pharmacia Biotech). The Western blot clearly showed that TAT-ZFP fusion proteins were detected inside the HEK 293 cells.

### EXAMPLE 3

### TAT-ZFP Mediated Regulation of Reporter Gene Activity and Endogenous Gene Expression

To confirm that transduced TAT-ZFP fusion proteins function as transcription factors within cells, we transfected cells with a series of firefly luciferase reporters containing ZFP-binding sequences specific for the TAT-ZFPs that we were testing. In this example, we used ZFPs that bind to the VEGF promoter. In U.S. Ser. No. 60/431,892, filed Dec. 9, 2003, we reported that these proteins could regulate the endogenous VEGF gene.

We prepared the transfected cells as follows. Human embryonic kidney 293 cells were maintained in Dulbecco's modified Eagle medium (DMEM) supplemented with 100 units/ml penicillin, 100 μg/ml streptomycin, and 10% fetal bovine serum (FBS). For the luciferase assay, 10⁴ cells/well were pre-cultured in a 96-well plate. 293 cells were transfected with 25 ng of the reporter plasmid using a LIPOFECTAMINE™ transfection kit (Life Technologies, Rockville, Md.). The reporter plasmid includes the native VEGF promoter fused to the luciferase gene in pGL3-basic (Promega).

We incubated the cells for 24 hours, and then contacted them with culture media that included lysates containing TAT-ZFP proteins for an additional 24 hours. The culture media either included 40 μg or 80 μg of lysate. After this 24-hour period, we assayed the cells for reporter expression with a Dual luciferase assay kit (Promega) using a TD-20/20 luminometer (Turner Designs Inc., Sunnyvale, Calif.).

The TAT-F475-KRAB lysate suppressed reporter activity 1.5 fold (when 40 μg was used) and 2.0 fold (80 μg) relative to control cultures transduced with a lysate of a corresponding TAT-only protein (i.e., a protein that does not include zinc finger domains). The observed suppression was due to sequence specific DNA binding, because lysates of the TAT-F83-KRAB, which has a different DNA binding specificity than TAT-F475-KRAB, did not suppress reporter activity. The chimeric zinc finger domain F83 used in this experiment served as negative control because it did not alter expression of the luciferase reporter that included native human VEGF promoter sequence in our previous work (Bae et al. Nat Biotechnol. 2003, 21(3):275-80). Similarly a lysate of TAT-mF475-KRAB did not suppress reporter activity. mF475 includes mutations (arginine to alanine) that disable DNA binding in the second and third zinc finger domains. These indicate that the regulation of reporter gene expression was due to specific zinc finger protein, and not by TAT protein transduction domain.

These results demonstrate that the tat domain of TAT-F475-KRAB efficiently transduces the protein into cells and that the transduced protein can regulate transcriptions its target genes within such cells in a sequence specific manner.

### EXAMPLE 4

### Transduction of Proteins that include a Transcriptional Regulatory Domain

We next asked whether proteins that include a transcriptional regulatory domain could also be transduced into cells. We prepared proteins that include a protein transduction domain, an array of zinc finger domains, and either the p65 transcriptional activator domain or the KRAB transcriptional repressor domain. The plamid encoding TAT-ZFP fusion proteins were expressed from E. coli BL21(DE3) transformants as indicated in example 1. We contacted these proteins to 293 cells transiently transfected with a luciferase reporter construct containing native human VEGF promoter sequence (Bae et al. Nat Biotechnol. 2003, 21(3):275-80) that the F475 zinc fingers recognize. Lysates of TAT-F475-p65 caused reporter activity to increase 2.5 fold (±0.48) compare to cells transduced with a control lysate (including a Tat protein that does not have zinc finger domains). Purified TAT-F121-KRAB caused reporter activity to decrease by 2.1 fold (±0.14).

### EXAMPLE 5

### Regulation of Endogenous Genes by Transduced DNA Binding Proteins

We also evaluated whether transduced DNA binding proteins could regulate endogenous VEGF mRNA expression. We added lysates that include the TAT-F475-KRAB protein or the TAT-F121-KRAB protein to 3×10⁵293 cell cultures (12-well culture plate) that were pre-cultured for 24 hours. At four hours after contact, the culture media containing the lysates was replaced with fresh media. The cells were harvested 24 hours after contact with the lysates. Total cellular RNA was extracted from the cells by preparing TRIZOL™-lysates according to the manufacturer's instructions (Life Technologies).

The reverse transcription reactions were performed with 4 μg total RNA using oligo-dT as the first-strand synthesis primer for mRNA and the MMLV reverse transcriptase provided in the SUPERSCRIPT™ first-strand synthesis system (Life Technologies). To analyze mRNA quantities, 1 μl each of the first-strand cDNAs generated from the RT reactions was amplified using VEGF-specific primers. The initial amounts of RNA were normalized to glyceraldehydes-3-phosphate dehydrogenase (GAPDH) mRNA concentrations that had been calculated by specific amplification using GAPDH-specific primers. The amplification of VEGF and GAPDH cDNAs was monitored and analyzed in real-time with a QUANTITECT™ SYBR kit (Qiagen, Valencia, Calif.) and ROTORGENE 2000™ real-time cycler (Corbett, Sydney, Australia). mRNA concentrations were quantified using serial dilution of the standards included in the reactions.

Both TAT-F475-KRAB and TAT-F121-KRAB suppressed endogenous VEGF mRNA level by 3.1 (±0.4) fold and 1.7 (±0.01) fold respectively compare to a control protein that includes the TAT domain but not a zinc finger domain. This result demonstrates that transduced ZFPs can function as artificial transcription factors within cells.

We also asked whether transduced TAT-ZFP proteins affect the regulation of other endogenous genes. We compared transcription profiles obtained using DNA microarray results for cells stably expressing F121-KRAB and a mutant variant mF121-KRAB. The mF121-KRAB (QSHT-ASHR-ADHT) includes two amino acid substitutions (Arg to Ala) relative to F121 (QSHT-RSHR-RDHT) at amino acid positions that contact DNA. This mutation is expected to alter or disrupt specificity of DNA binding.

The transcriptional profiles confirmed that VEGF mRNA was down regulated by F121-KRAB. In addition, the profiles demonstrated that F121-KRAB caused activation of a group of genes including the Annexin A3 and Cyclin A gene. The transcriptional profile of mF121-KRAB treated cells did not show activation of Annexin A3 and Cyclin A gene activation nor a change in VEGF mRNA expression. The same results were obtained by RT-PCR. These results demonstrate, for the first time, that the chimeric zinc finger proteins fused to the TAT PTD and expressed in E. Coli, can be transduced successfully into mammalian cells and can function as transcriptional regulators after transduction.

### EXAMPLE 6

### TAT-ZFP Fusion Protein Functioned as Transcriptional Activator and Repressor According to Effector Domains

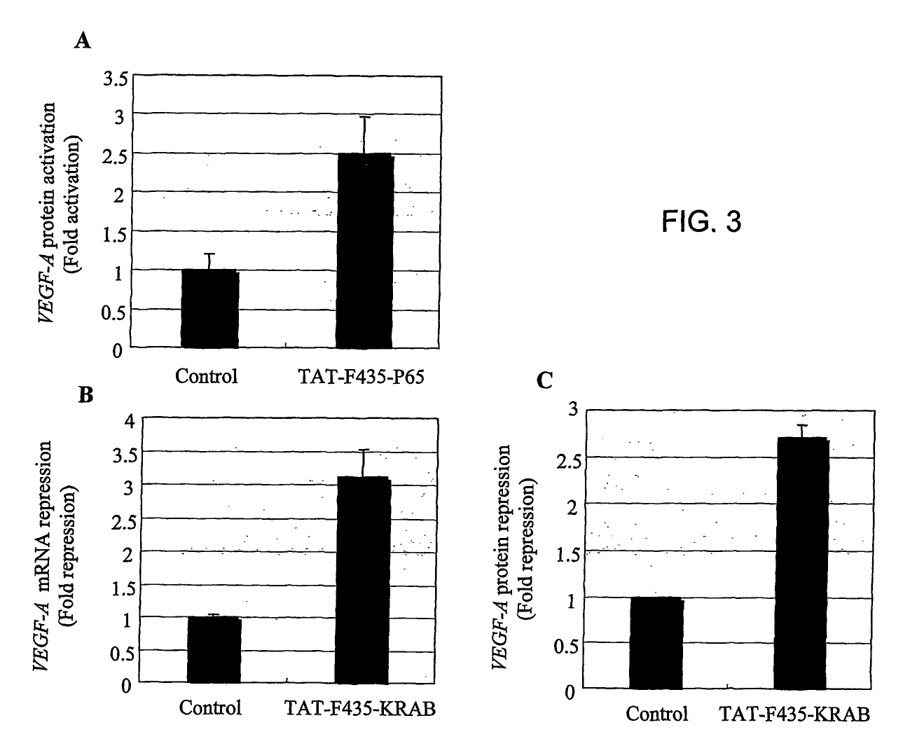

We observed that a given DNA binding domain functioned both as an activator and as repressor of transcription, depending the character of the effector domain that is operably linked to the DNA binding domain. For example, TAT-F435 was able to both activate and suppress endogenous VEGF-A expression depending on whether it was fused to the p65 activation domain or the KRAB repression domain. The BL21 (DE3)LysS bacteria (Novagen) were transformed with plasmids encoding TAT-F435-KRAB or TAT-F435-p65. The fusion proteins TAT-F435-KRAB or TAT-F435-p65 were expressed from transformants. See example 1. In the experiments with TAT-F435-p65, we treated 293 cells with 100 ug of bacterial lysate that contained TAT-F435-p65 protein due to its low expression level. The cells were incubated for 3 hours with lysate then the culture medium was changed with fresh medium. However this was sufficient to increase VEGF expression 2.5 (0.5) fold measured by secreted VEGF at 48 hours after transduction compared to cells treated with bacterial lysate that do not express TAT-F435-p65 (See FIG. 3 A).

In separate experiments, 400 ug/ml of purified TAT-F435-KRAB protein was exposed to 293 cells (12-well plate) for 3 hours. The cells were incubated further 48 h in fresh culture media. The culture supernatants and cells were harvested to analyze VEGF protein production and VEGF mRNA amount. We observed that the TAT-F435-KRAB repressed VEGF mRNA level by 3.1 (±0.35) fold measured by RT-PCR method (normalized to GAPDH mRNA quantity) compared to PBS treated control culture (See FIG. 3 B). The VEGF production was also decreased by 2.7 (±0.2) fold in the culture treated with TAT-F435-KRAB compared to control culture treated with PBS (See FIG. 3 C).

### EXAMPLE 7

### TAT-F435-KOX Protein Reduces VEGF-A Expression in the Human Lung Cancer Cell Line H460

VEGF-A is overexpressed in highly vascularizing tumorigenic lines, such as the non-small cell lung cancer cell line H460 and the human colon cancer lines HCT116 and HM-7. Expression of VEGF-A is 5- to 10-fold higher in these cells compared to its expression in HEK293F cells. To test the general applicability of transducible zinc finger proteins as cancer therapeutics, we evaluated the ability of TAT-F435-KOX protein to override the cancer-specific transcriptional circuitry that drives aberrant VEGF-A overexpression in H460 cells. Our observations also confirm that the TAT-ZFP-KOX and related configurations can be used to design transducible proteins to regulate endogenous genes.

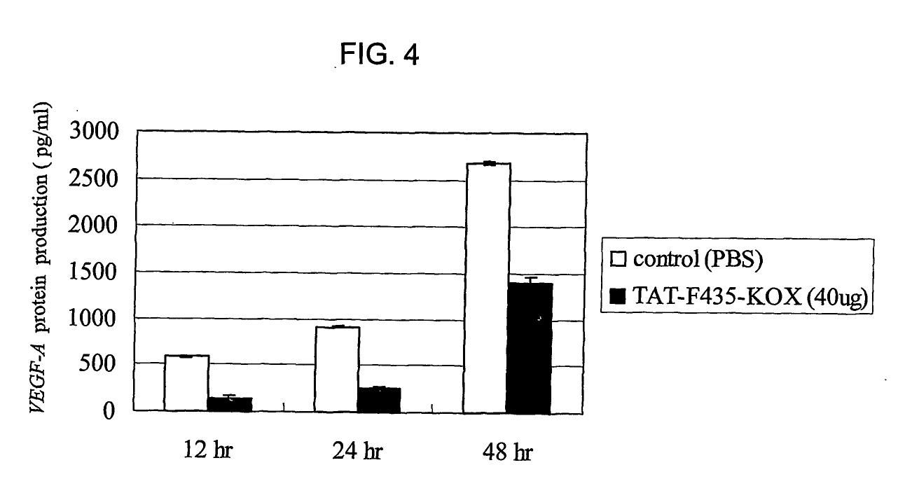

We treated H460 cells with the TAT-F435-KOX protein. TAT-F435-KOX protein reduced the expression level of VEGF-A protein, about 3-fold compared to control. The H460 cells were exposed to 40 μg/well of TAT-F435-KOX protein for three hours. Subsequently, the concentration of secreted VEGF-A protein was measured at various time points. VEGF-A concentration in media surrounding the treated cells was reduced to about 3.8-fold relative to untreated controls 12 hours after transduction, 3.4-fold 24 hours after, and 1.9-fold 48 hours after. See FIG. 4. These results indicate that TAT-F435-KOX can repress expression of VEGF-A in a cancer cell. The regulatory effect of TAT-F435-KOX continued for at least 48 hours after a single in-vitro treatment. TAT-F435-KOX had a similar effect in a H460 cell line when used at a dose of 10 μg/well.

### EXAMPLE 8

### Stability of TAT-F435-KOX Protein

We studied the stability of TAT-F435-KOX protein in a cell-free culture media. The TAT-F435-KOX protein was purified with an Ni-NTA column. Then, the protein was incubated in DMEM (10% FBS, 1% NEAA) at 37° C. The mixture was evaluated for TAT-F435-KOX protein at various time points by western blot analysis with an HA-specific antibody.

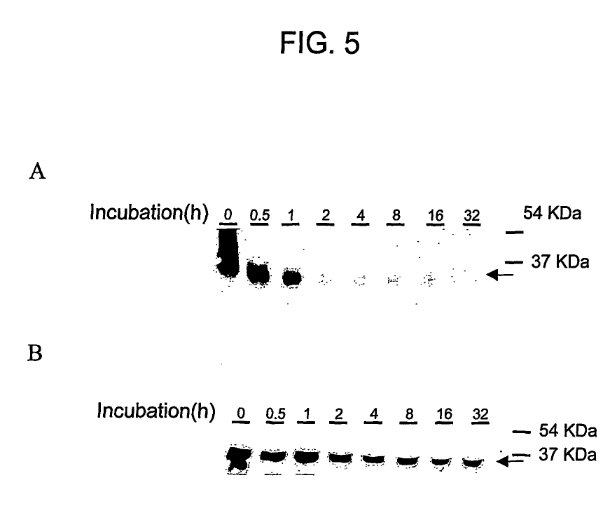

The western blot showed that the TAT-F435-KOX protein (purified using a single column step with the Ni-NTA column) was rapidly cleared and was unlikely to maintain its activity for prolonged duration. See FIG. 5, A. To investigate whether the clearance of the protein purified by this method was due to protease activity, we treated TAT-F435-KOX protein with protease inhibitor cocktail (P8849, Sigma) before addition to culture media. The treatment of protease inhibitors significantly improved the stability of TAT-F435-KOX protein in culture condition. See FIG. 5, B.

We compared these observations to the behavior of the same protein purified using at least two columns. In this case, after the NI-NTA affinity column, the protein was further purified on an ion-exchange column.

We then evaluated the stability of the protein in H460 human colon cancer cells using about 50000 cells and 80 μl of TAT-F435-Kox. The cells were treated with this second preparation of TAT-F435-KOX protein for three hours. The media was replaced with fresh growth media. Then the cells were harvested at various time points after the treatment. Control cells were incubated with TAT-F435-KOX protein for less than five minutes before harvesting.

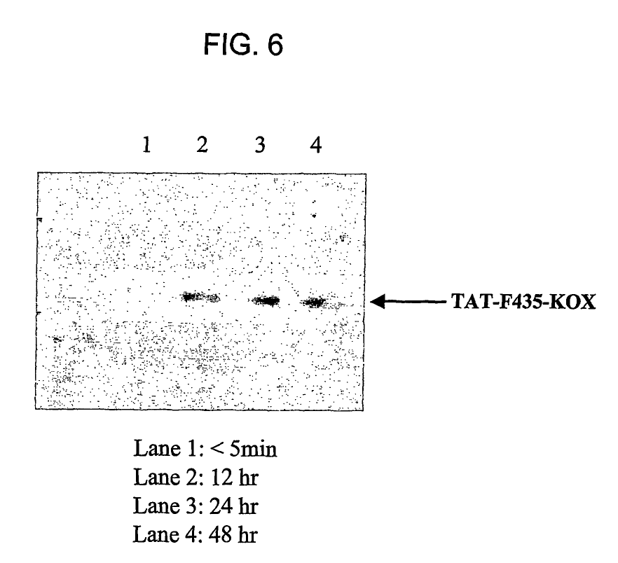

To ensure that the protein detected by western blot was not residual TAT-F435-KOX protein that did not enter cells, but remained in the media, cells were trypsinized to remove proteins bound to cell surface and washed extensively before preparing cell lysate. 80 μg of each lysate was electrophoresed on an SDS-PAGE gel, transfer to a HYBOND-P™ membrane (Amersham Pharmacia Biotech) and contacted with antibodies for detection. Intact TAT-F435-KOX protein was detected in these cell lysates. In particular, the protein (TAT-F435-KOX) purified consecutively by Ni-NTA affinity column and an ion-exchange column was observed to be stable for at least 48 hours within cells. See FIG. 6.

The TAT-F435-KOX proteins (and other transducible proteins described herein) can be expressed and purified from inclusion bodies or as a soluble protein. We compared the properties of TAT-F435-KOX protein purified from inclusion bodies to the same protein produced using a soluble expression system. Zinc finger proteins from inclusion bodies or other systems can be refolded in the presence of zinc. For example, the proteins can be denatured (e.g., in a chaotrope such as urea) and then dialyzed against a buffer with zinc, e.g., PBS (plus 20 μM ZnCl₂, 1 mM DTT). The dialysis can be carried out with buffer (4 liter) exchange every 2 hours, e.g., with total 4 exchanges. The procedures are typically done at 4° C.

For soluble expression, a nucleic acid sequence encoding TAT-F435-KOX was subcloned into a modified pET43.1B plasmid (Novagen). This plasmid includes a sequence encoding a hexa-histidine tag. The protein was expressed as a fusion protein of His6-NusA-TAT-F435-KOX in E. coli strain BL21(DE3). The fusion protein was purified on an Ni-NTA column. The NusA domain was removed by thrombin cleavage since there is a cognate thrombin cleavage site N-terminal to the TAT domain. The released TAT-F435-KOX can be further purified on an ion exchange column.

293F cells were treated with TAT-F435-KOX proteins purified from inclusion bodies and the same protein purified from the soluble expression system. Both proteins repressed production of VEGF-A protein. However, the difference in the degree of repression was not significant.

A number of embodiments of the invention have been described. Nevertheless, it will be understood that various modifications may be made without departing from the spirit and scope of the invention. Accordingly, other embodiments are within the scope of the following claims.

What is claimed is:

1. A chimeric protein comprising:
a) a plurality of zinc finger domains; and
b) a heterologous protein transduction domain, effective for translocating the protein across a cellular membrane.

2. The chimeric protein of claim 1, wherein the protein transduction domain is N-terminal or C-terminal to the plurality of zinc finger domains.

3. The chimeric protein of claim 1, wherein the plurality of zinc finger domains comprises 3 to 6 zinc finger domains.

4. The chimeric protein of claim 1, wherein the chimeric protein specifically binds to a site in the VEGF-A gene and can regulate transcription of the VEGF-A gene in a cell.

5. The chimeric protein of claim 1, wherein the chimeric protein specifically binds to a site in a gene and can regulate transcription of the gene in a cell, the gene encoding a protein selected from the group consisting of: jun B proto-oncogene, protein kinase C, lectin, brain-specific Na-dependent inorganic phosphate cotransporter, cellular retinoic acid-binding protein 1, cellular retinoic acid-binding protein 2, cadherin 13, H-cadherin (heart), vascular endothelial growth factor (VEGF-A), pigment epithelium-derived factor (PEDF), differentiation-related gene-1 (Drg-1), Transcription factor E2F, Early growth response-1 (EGR-1), protein tyrosine phosphatases 1B (PTP-1B), A20, Fas, melanoma differentiation associated gene-7 (MDA-7), presenilin-1 (PS-1), angiotensin converting enzyme, Angiopoietin-2, b-secretase (BACE1), mmp3, checkpoint with forkhead associated and ring finger (CHFR), peroxisome proliferator-activated receptor gamma (PPAR-gamma), TNF-related apoptosis-inducing ligand (TRAIL), Ku-80, ataxia-telangiectasia mutated (ATM), BRCA, CC-chemokine receptor 5 (CCR5), brain-derived neurotrophic factor (BDNF), tumor necrosis factor alpha-induced protein-3 (TNFAIP3) (A20), c-myc, Hypoxia-inducible factor-1 alpha (HIF-1 alpha), caspase-3, intercellular adhesion molecule type I (ICAM-1), angiotensin II receptor 1 (AT-1R), platelet-derived growth factor, insulin-like growth factor-I and -II, nerve growth factor, aFGF, bFGF, epidermal growth factor, TGF-α, TGF-β, erythropoietin, thrombopoietin, mucins, growth hormone, proinsulin, insulin A-chain, insulin B-chain, parathyroid hormone, thyroid stimulating hormone, thyroxine, follicle stimulating hormone, calcitonin, factor VIII, hematopoietic growth factor, enkephalinase, Mullerian-inhibiting substance, gonadotropin-associated peptide, tissue factor protein, inhibin, activin, interferon-α, interferon-β, interferon-γ, M-CSF, GM-CSF, G-CSF, IL-1, IL-2, IL-3, IL-4. IL-12, and IL-13.

6. The chimeric protein of claim 1, wherein the plurality of zinc finger domains includes domains whose DNA contacting residues correspond to DNA-contacting residues of a set of motifs in a row of Table 2.

7. The chimeric protein of claim 1, wherein the plurality of zinc finger domains includes domains whose DNA contacting residues correspond to DNA-contacting residues of a set of motifs selected from the group consisting of: mQSHR-mRDHT-mRSNR; mQSHT-mRSHR-mPDHT; mQSHR-mRDHT-mRSHR; mRSHR-mRDHT-mVSNV; mQSHV-mRDHR-mRDHT; mRDER-mQSSR-mQSHT-mRSNR; in DSAR-mRSNR-mRDHT-mVSSR; mQSHT-mDSAR-mRSNR-mRDHT; mRDHT-mVSNV-mQSHT-mDSAR; mRSHR-mDSCR-mQSHT-mDSCR; mQSNR-mQSHR-mRDHT-mRSNR; mCSNR-mRDHT-nRSNR-mRSHR; mRSHR-mQSHT-mRSHR-mRDER; mQSNR-mRSHR-mQSSR-mRSHR; mQSHT-tnDSCR-mRDHT-mCSNR; mQSHT-mWSNR-mRSHR-mWSNR; and mVSNV-mRSHR-mRDER-mQSNV

8. The chimeric protein of claim 1, further comprising a nuclear localization signal.

9. The chimeric protein of claim 1, wherein at least one of the zinc finger domains of the plurality has the sequence of a naturally occurring zinc finger domain.

10. The chimeric protein of claim 9, wherein at least one of the zinc finger domains of the plurality is human.

11. The chimeric protein of claim 1, wherein the protein transduction domain comprises a viral sequence or a human sequence.

12. The chimeric protein of claim 1, wherein the protein transduction domain comprises an HIV tat protein transduction domain, HSV VP22 protein, or Antennapedia homeodomain.

13. The chimeric protein of claim 12, wherein the HIV tat protein transduction domain comprises the amino acid sequence: YGRKKRRQRRR (SEQ ID NO: 1).

14. The chimeric protein of claim 1, wherein the protein transduction domain comprises a modified or synthetic protein transduction domain.

15. The chimeric protein of claim 1, wherein the protein transduction domain comprises a modified tat protein transduction domain having the amino acid sequence of any one of SEQ ID NOs: 69 to 72, a polyarginine oligopeptide consisting of 6 to 12 arginine residues, or HN1 synthetic peptide having the amino acid sequence of SEQ ID NO: 4.

16. The chimeric protein of claim 1, wherein the chimeric protein can regulate at least one endogenous gene in a cell after the protein is contacted with a membrane of the cell.

17. The chimeric protein of claim 1, wherein the chimeric protein can regulate transcription of at least one endogenous gene in a cell, but fewer than 1% of the genes in the cell.

18. The chimeric protein of claim 1, wherein the chimeric protein can regulate transcription of at least one endogenous gene in a cell, but fewer than 0.01% of the genes in the cell.

19. The chimeric protein of claim 1, wherein the chimeric protein can translocate from the extracellular milieu into a mammalian cell in the absence of a cell permeabilization reagent.

20. The chimeric protein of claim 1, wherein the protein transduction domain and the plurality of zinc finger domains are components of the same polypeptide chain.

21. The chimeric protein of claim 1, further comprising a cell targeting domain.

22. The chimeric protein of claim 21, wherein the cell targeting domain comprises an immunoglobulin variable domain, a growth factor, a cell binding domain of a viral protein, or a cell binding domain of an extracellular protein.

23. The chimeric protein of claim 1, further comprising a cell surface protein binding domain.

24. The chimeric protein of claim 1, further comprising a purification handle.

25. The chimeric protein of claim 24, wherein the purification handle comprises an amino acid sequence that can chelate metal.

26. The chimeric protein of claim 1, wherein each zinc finger domain of the plurality is bound to a zinc atom.

27. The chimeric protein of claim 1, further comprising a transcription activation domain.

28. The chimeric protein of claim 27, wherein the transcription activation domain comprises p65 or VP16 activation domain.

29. The chimeric protein of claim 1, further comprising a transcription repression domain.

30. The chimeric protein of claim 29, wherein the transcription repression domain comprises Kid or KOX repression domain.

31. The chimeric protein of claim 1, wherein the protein can be transduced into at least 50% of cultured human embryonic kidney (HEK) 293 cells in an assay in which the cells are at 3×10⁵ cells/ml and the protein is present in the extracellular medium at concentration of 100 micrograms/ml.

32. The chimeric protein of claim 1, wherein the chimeric protein is stable for at least 0.5 hours in human tissue culture cells.

33. A pharmaceutical composition comprising:
the chimeric protein of claim 1;
a pharmaceutically acceptable carrier.

34. The composition of claim 33, further comprising a reducing agent in an amount effective to decrease disulfide bond formation between cysteine residues of the zinc finger domain of the protein.

35. The composition of claim 34, wherein the reducing agent comprises glutathione or DTT.

36. The composition of claim 34, wherein the protein is stable in cell culture media for at least 12 hours when the composition is combined with cell culture media.

37. The composition of claim 34, further comprising between about 1 μM and 500 μM zinc chloride.

38. The composition of claim 33, which is used for the treatment of a subject having or being suspected of having a neoplastic disorder, an inflammatory disorder or an angiogenesis-based disorder, and the chimeric protein further comprising an effector domain such that the chimeric protein regulates VEGF-A transcription in cells of the subject.

39. A nucleic acid comprising a coding sequence that encodes a polypeptide that comprises a) a zinc finger domain; and b) a heterologous protein transduction domain, located N-terminal to the zinc finger domain.

40. A method of altering gene expression in a cell of a subject, the method comprising:
administering, to a subject, a chimeric DNA binding protein that comprises a plurality of zinc finger domains and a heterologous protein transduction domain, the DNA binding protein being able to regulate transcription of an endogenous gene in a cell of the subject.

41. The method of claim 40, wherein the chimeric protein is administered as a first dose, and the method further comprises administering a second dose of the chimeric protein to the subject.

42. The method of claim 41, wherein the first and second dose are separated by at least about 48 hours.

43. The method of claim 41, wherein the first dose is at least 25% less than the second dose.

44. The method of claim 40, wherein the endogenous gene is VEGF-A.

45. The method of claim 44, wherein the subject has or is suspected of having a neoplastic disorder, an inflammatory disorder or an angiogenesis-based disorder, and the DNA binding protein further comprises an effector domain such that the DNA binding protein regulates VEGF-A transcription in cells of the subject.

46. A method of altering gene expression in a eukaryotic culture cell, the method comprising:
contacting the cell with a dose of a chimeric DNA binding protein that comprises a plurality of zinc finger domains and a protein transduction domain, the protein being able to regulate transcription of an endogenous gene in a cell, wherein the dose is effective to regulate transcription of the endogenous gene for at least 48 hours.

47. The method of claim 46, further comprising, at least 48 hours after the contacting, contacting the cell with a second dose of the DNA binding protein.

48. The method of claim 46, wherein the endogenous gene is VEGF-A.

49. A eukaryotic cell that contains an exogenous polypeptide, but not a nucleic acid that encodes the exogenous polypeptide, wherein the exogenous polypeptide comprises a plurality of zinc finger domains and a protein transduction domain that is heterologous to the DNA binding domain, the exogenous polypeptide being functional to regulate transcription of a selected subset of endogenous genes in the cell for at least 12 hours after introduction of the exogenous polypeptide into the cell.

50. The eukaryotic cell of claim 49, wherein the plurality of zinc finger domains comprises a naturally occurring zinc finger domain.

51. The eukaryotic cell of claim 49, wherein the plurality of zinc finger domains comprises a human zinc finger domain.

52. The eukaryotic cell of claim 49, wherein exogenous polypeptide remains functional in the cell for at least 48 hours.

53. A method of preparing a transducible DNA binding polypeptide, the method comprising
providing a host cell that contains a nucleic acid comprising
1) a coding sequence that encodes a polypeptide that comprises a) a zinc finger domain; and b) a heterologous protein transduction domain, and
2) a promoter operably linked to the coding sequence;
expressing the nucleic acid in the host cell under conditions in which the polypeptide is synthesized; and
isolating the polypeptide from the host cell or from medium surrounding the host cell.

54. The method of claim 53, wherein the isolating comprises purifying inclusion bodies.

55. The method of claim 53, wherein the isolating comprises affinity chromatography and ion exchange chromatography.

56. The method of claim 53, further comprising combining the isolated polypeptide with a pharmaceutically acceptable carrier, thereby preparing a pharmaceutical composition.

57. A method of altering gene expression in a eukaryotic cell, the method comprising:
contacting a eukaryotic cell with a chimeric DNA binding protein that comprises a plurality of zinc finger domains and a protein transduction domain, the protein being able to regulate transcription of an endogenous gene in the cell.

58. The method of claim 57, wherein the eukaryotic cell is a mammalian cell.

59. The method of claim 57, wherein the eukaryotic cell is a human cell.

60. The method of claim 57, wherein the cell is a culture cell.

61. The method of claim 57, wherein the cell is obtained from a subject or resides in a subject.

62. The method of claim 61, wherein the subject is human.

63. The method of claim 57, wherein the chimeric DNA binding protein comprises a plurality of zinc finger domains.

64. The method of claim 57, wherein the protein transduction domain comprises a viral sequence or a human sequence.

65. The method of claim 64, wherein the protein transduction domain comprises an HIV tat protein transduction domain, HSV VP22 protein, or Antennapedia homeodomain.

66. The method of claim 65, wherein the HIV tat protein transduction domain comprises the amino acid sequence: YGRKKRRQRRR (SEQ ID NO:1).

67. The method of claim 57, wherein the protein transduction domain comprises a modified or synthetic protein transduction domain.

68. The method of claim 57, wherein the protein transduction domain comprises a modified tat protein transduction domain having the amino acid sequence of any one of SEQ ID NOs: 69 to 72, apolyarginine oligopeptide consisting of 6 to 12 arginine residues, or HN1 synthetic peptide having the amino acid sequence of SEQ ID NO: 4.

69. The method of claim 57, wherein the protein transduction domain and the plurality of zinc finger domains are components of the same polypeptide chain.

70. The method of claim 57, wherein the chimeric DNA binding protein further comprises a cell targeting domain.

71. The method of claim 70, wherein the cell targeting domain comprises an immunoglobulin variable domain, a growth factor, a cell binding domain of a viral protein, or a cell binding domain of an extracellular protein.

72. The method of claim 57, wherein the endogenous gene is a gene encoding a protein selected from the group consisting of: jun B proto-oncogene, protein kinase C, lectin, brain-specific Na-dependent inorganic phosphate cotransporter, cellular retinoic acid-binding protein 1, cellular retinoic acid-binding protein 2, cadherin 13, H-cadherin (heart), vascular endothelial growth factor (VEGF-A), pigment epithelium-derived factor (PEDF), differentiation-related gene-I (Drg-1), Transcription factor E2F, Early growth response-I (EGR-1), protein tyrosine phosphatases 1B (PTP-1B), A20, Fas, melanoma differentiation associated gene-7 (MDA-7), presenilin-1 (PS-1), angiotensin converting enzyme, Angiopoietin-2, b-secretase (BACEI), mmp3, checkpoint with forkhead associated and ring finger (CHFR), peroxisome proliferator-activated receptor gamma (PPAR-gamma), TNF-related apoptosis-inducing ligand (TRAIL), Ku-80, ataxia-telangiectasia mutated (ATM), BRCA, CC-chemokine receptor 5 (CCR5), brain-derived neurotrophic factor (BDNF), tumor necrosis factor or alpha-induced protein-3 (TNFAIP3) (A20), c-myc, Hypoxia-inducible factor-1 alpha (HIF-1alpha), caspase-3, intercellular adhesion molecule type I (ICAM-1), angiotensin II receptor 1 (AT-1R), platelet-derived growth factor, insulin-like growth factor-I and -II, nerve growth factor, aFGF, bFGF, epidermal growth factor, TGF-α, TGF-β, erythropoietin, tbrombopoietin, mucins, growth hormone, proinsulin, insulin A-chain, insulin B-chain, parathyroid hormone, thyroid stimulating hormone, thyroxine, follicle stimulating hormone, calcitonin, factor VIII, hematopoietic growth factor, enkephalinase, Mullerian-inhibiting substance, gonadotropin-associated peptide, tissue factor protein, inlibin, activin, interferon-α, interferon-β, interferon-γ, M-CSF, GM-CSF, G-CSF, IL-1, IL-2, IL-3, IL-4. IL-12, and IL-13.

73. The method of claim 57, wherein the chimeric DNA binding protein specifically binds to a site within 1000, 500, 300, or 100 base pairs of the transcriptional start site.

74. The method of claim 57, wherein the chimeric DNA binding protein specifically binds to a site within 1000, 500, 300, or 100 base pairs of the TATA box.

75. The method of claim 57, wherein the chimeric DNA binding protein specifically binds to a site within 20 base pairs or a site that overlaps with a site bound by a naturally occurring transcription factor in a regulatory region of the endogenous gene.

76. The method of claim 57, wherein the chimeric DNA binding protein increases expression of the endogenous gene.

77. The method of claim 57, wherein the chimeric DNA binding protein decreases expression of the endogenous gene.

78. The method of claim 57, wherein the cell is a neoplastic cell.

79. A host cell that comprises a nucleic acid comprising a coding sequence and a promoter operably linked to the coding sequence, wherein the coding sequence encodes a polypeptide that comprises a signal sequence, a plurality of zinc finger domains, and a protein transduction domain.

80. A method of altering expression of an endogenous gene, the method comprising:
introducing, into one or more cells, a nucleic acid comprising a coding sequence and a promoter operably linked to the coding sequence, wherein the coding sequence encodes a chimeric that comprises a signal sequence, a plurality of zinc finger domains, and a protein transduction domain, such that the cells express the nucleic acid and produce and secrete the chimeric protein, whereby the chimeric protein can enter other cells and regulate expression of an endogenous gene in such cells.

81. The method of claim 80, wherein the cells into which the nucleic acid is introduced are in a subject.


SEQUENCE LISTING (72 sequences)

SEQ ID NO: 1 - protein, 11 aa, Human immunodeficiency virus
YGRKKRRQRRR

SEQ ID NO: 2 - protein, 16 aa, Drosophila melanogaster
AKIWFQNRRMKWKKEN

SEQ ID NO: 3 - protein, 34 aa, Herpes simplex virus
DAATATRGRSAASRPTERPRAPARSASRPRRPVE

SEQ ID NO: 4 - protein, 12 aa, Artificial Sequence
  Description of Artificial Sequence Synthetic peptide
TSPLNIHNGQKL

SEQ ID NO: 5 - protein, 26 aa, Artificial Sequence
  Description of Artificial Sequence Synthetic peptide
CXXXXXCXXXXXXXXXXXXHXXXXXH

SEQ ID NO: 6 - protein, 26 aa, Artificial Sequence
  Description of Artificial Sequence Synthetic peptide
CXXXXXCXXXXXRXDEXXRHXXXXXH

SEQ ID NO: 7 - protein, 260 aa, Homo sapiens
YLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTAQRPPDPAPAPLGAPGLPNGLLSGDEDFSSIADMDFSALLSQ

SEQ ID NO: 8 - protein, 127 aa, Saccharomyces cerevisiae
NFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKEISMAYPYDVPDYAS

SEQ ID NO: 9 - protein, 90 aa, Saccharomyces cerevisiae
NSASSSTKLDDDLGTAAAVLSNMRSSPYRTHDKPISNVNDMNNTNALGVPASRPHSSSFPSKGVLRPILLRIHNSEQQPIFESNNSTACI

SEQ ID NO: 10 - protein, 63 aa, Homo sapiens
VSVTFEDVAVLFTRDEWKKLDLSQRSLYREVMLENYSNLASMAGFLFTKPKVISLLQQGEDPW

SEQ ID NO: 11 - protein, 96 aa, Homo sapiens
DAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 12 - protein, 23 aa, Homo sapiens
YKCKQCGKAFGCPSNLRRHGRTH

SEQ ID NO: 13 - protein, 23 aa, Homo sapiens
YQCNICGKCFSCNSNLHRHQRTH

SEQ ID NO: 14 - protein, 23 aa, Homo sapiens
YSCGICGKSFSDSSAKRRHCILH

SEQ ID NO: 15 - protein, 23 aa, Homo sapiens
YTCSDCGKAFRDKSCLNRHRRTH

SEQ ID NO: 16 - protein, 23 aa, Homo sapiens
YKCKECGKAFNHSSNFNKHHRIH

SEQ ID NO: 17 - protein, 23 aa, Homo sapiens
FKCPVCGKAFRHSSSLVRHQRTH

SEQ ID NO: 18 - protein, 24 aa, Homo sapiens
YRCKYCDRSFSISSNLQRHVRNIH

SEQ ID NO: 19 - protein, 23 aa, Homo sapiens
YECDHCGKAFSIGSNLNVHRRIH

SEQ ID NO: 20 - protein, 23 aa, Homo sapiens
YGCHLCGKAFSKSSNLRRHEMIH

SEQ ID NO: 21 - protein, 23 aa, Homo sapiens
YKCKECGQAFRQRAHLIRHHKLH

SEQ ID NO: 22 - protein, 23 aa, Homo sapiens
YKCHQCGKAFIQSFNLRRHERTH

SEQ ID NO: 23 - protein, 23 aa, Homo sapiens
FQCNQCGASFTQKGNLLRHIKLH

SEQ ID NO: 24 - protein, 23 aa, Homo sapiens
YACHLCGKAFTQSSHLRRHEKTH

SEQ ID NO: 25 - protein, 23 aa, Homo sapiens
YKCGQCGKFYSQVSHLTRHQKIH

SEQ ID NO: 26 - protein, 23 aa, Homo sapiens
YACHLCGKAFTQCSHLRRHEKTH

SEQ ID NO: 27 - protein, 23 aa, Homo sapiens
YACHLCAKAFIQCSHLRRHEKTH

SEQ ID NO: 28 - protein, 23 aa, Homo sapiens
YVCRECGRGFRQHSHLVRHKRTH

SEQ ID NO: 29 - protein, 23 aa, Homo sapiens
YKCEECGKAFRQSSHLTTHKIIH

SEQ ID NO: 30 - protein, 23 aa, Homo sapiens
YECDHCGKSFSQSSHLNVHKRTH

SEQ ID NO: 31 - protein, 23 aa, Homo sapiens
YMCSECGRGFSQKSNLIIHQRTH

SEQ ID NO: 32 - protein, 23 aa, Homo sapiens
YKCEECGKAFTQSSNLTKHKKIH

SEQ ID NO: 33 - protein, 23 aa, Homo sapiens
FECKDCGKAFIQKSNLIRHQRTH

SEQ ID NO: 34 - protein, 23 aa, Homo sapiens
YVCRECRRGFSQKSNLIRHQRTH

SEQ ID NO: 35 - protein, 23 aa, Homo sapiens
YECEKCGKAFNQSSNLTRHKKSH

SEQ ID NO: 36 - protein, 23 aa, Homo sapiens
YECNTCRKTFSQKSNLIVHQRTH

SEQ ID NO: 37 - protein, 23 aa, Homo sapiens
YVCSKCGKAFTQSSNLTVHQKIH

SEQ ID NO: 38 - protein, 23 aa, Homo sapiens
YKCDECGKNFTQSSNLIVHKRIH

SEQ ID NO: 39 - protein, 23 aa, Homo sapiens
YECDVCGKTFTQKSNLGVHQRTH

SEQ ID NO: 40 - protein, 23 aa, Homo sapiens
YECVQCGKGFTQSSNLITHQRVH

SEQ ID NO: 41 - protein, 23 aa, Homo sapiens
YKCPDCGKSFSQSSSLIRHQRTH

SEQ ID NO: 42 - protein, 23 aa, Homo sapiens
YECQDCGRAFNQNSSLGRHKRTH

SEQ ID NO: 43 - protein, 23 aa, Homo sapiens
YECNECGKFFSQSSSLIRHRRSH

SEQ ID NO: 44 - protein, 23 aa, Homo sapiens
YKCEECGKAFNQSSTLTRHKIVH

SEQ ID NO: 45 - protein, 23 aa, Homo sapiens
YECNECGKAFAQNSTLRVHQRIH

SEQ ID NO: 46 - protein, 23 aa, Homo sapiens
YECHDCGKSFRQSTHLTQHRRIH

SEQ ID NO: 47 - protein, 23 aa, Homo sapiens
YECHDCGKSFRQSTHLTRHRRIH

SEQ ID NO: 48 - protein, 23 aa, Homo sapiens
HKCLECGKCFSQNTHLTRHQRTH

SEQ ID NO: 49 - protein, 25 aa, Homo sapiens
YVCDVEGCTWKFARSDELNRHKKRH

SEQ ID NO: 50 - protein, 25 aa, Homo sapiens
YHCDWDGCGWKFARSDELTRHYRKH

SEQ ID NO: 51 - protein, 25 aa, Homo sapiens
YRCSWEGCEWRFARSDELTRHFRKH

SEQ ID NO: 52 - protein, 25 aa, Homo sapiens
FSCSWKGCERRFARSDELSRHRRTH

SEQ ID NO: 53 - protein, 25 aa, Homo sapiens
FACSWQDCNKKFARSDELARHYRTH

SEQ ID NO: 54 - protein, 25 aa, Homo sapiens
YHCNWDGCGWKFARSDELTRHYRKH

SEQ ID NO: 55 - protein, 24 aa, Homo sapiens
FLCQYCAQRFGRKDHLTRHMKKSH

SEQ ID NO: 56 - protein, 23 aa, Homo sapiens
FQCKTCQRKFSRSDHLKTHTRTH

SEQ ID NO: 57 - protein, 23 aa, Homo sapiens
FACEVCGVRFTRNDKLKIHMRKH

SEQ ID NO: 58 - protein, 25 aa, Homo sapiens
YVCDVEGCTWKFARSDKLNRHKKRH

SEQ ID NO: 59 - protein, 23 aa, Homo sapiens
YKCMECGKAFNRRSHLTRHQRIH

SEQ ID NO: 60 - protein, 23 aa, Homo sapiens
YICRKCGRGFSRKSNLIRHQRTH

SEQ ID NO: 61 - protein, 23 aa, Homo sapiens
YLCSECDKCFSRSTNLIRHRRTH

SEQ ID NO: 62 - protein, 23 aa, Homo sapiens
YECKECGKAFSSGSNFTRHQRIH

SEQ ID NO: 63 - protein, 23 aa, Homo sapiens
YECDHCGKAFSVSSNLNVHRRIH

SEQ ID NO: 64 - protein, 23 aa, Homo sapiens
YTCKQCGKAFSVSSSLRRHETTH

SEQ ID NO: 65 - protein, 23 aa, Homo sapiens
YECNYCGKTFSVSSTLIRHQRIH

SEQ ID NO: 66 - protein, 23 aa, Homo sapiens
YRCEECGKAFRWPSNLTRHKRIH

SEQ ID NO: 67 - protein, 83 aa, Artificial Sequence
  Description of Artificial Sequence Synthetic peptide
YKCGQCGKFYSQVSHLTRHQKIHTGEKPFQCKTCQRKFSRSDHLKTHTRTHTGEKPYICRKCGRGFSRKSNLIRHQRTHTGEK

SEQ ID NO: 68 - protein, 83 aa, Artificial Sequence
  Description of Artificial Sequence Synthetic peptide
YKCEECGKAFRQSSHLTTHKIIHTGEKPYKCMECGKAFNRRSHLTRHQRIHTGEKPFQCKTCQRKFSRSDHLKTHTRTHTGEK

SEQ ID NO: 69 - protein, 11 aa, Artificial Sequence
  Description of Artificial Sequence Synthetic peptide
YARKARRQARR

SEQ ID NO: 70 - protein, 11 aa, Artificial Sequence
  Description of Artificial Sequence Synthetic peptide
YARAARRAARR

SEQ ID NO: 71 - protein, 11 aa, Artificial Sequence
  Description of Artificial Sequence Synthetic peptide
YARAARRAARA

SEQ ID NO: 72 - protein, 11 aa, Artificial Sequence
  Description of Artificial Sequence Synthetic peptide
YARAAARQARA